(19) United States
(12) Patent Application Publication
(10) Pub. No.: US 2025/0057591 A1
(43) Pub. Date: Feb. 20, 2025

(54) SYSTEMS AND METHODS FOR ELECTROGRAM SET DETERMINATION

(71) Applicant: KARDIUM INC., Burnaby (CA)

(72) Inventors: Daniel Martin REINDERS, Surrey (CA); Daniel Robert WEINKAM, Coquitlam (CA); Justin Aaron MICHAEL, Vancouver (CA)

(21) Appl. No.: 18/799,381

(22) Filed: Aug. 9, 2024

Related U.S. Application Data
(60) Provisional application No. 63/519,731, filed on Aug. 15, 2023.

(51) Int. Cl. A61B 18/14; A61B 17/00; A61B 18/00

(52) U.S. Cl. CPC A61B 18/1492; A61B 2017/00199; A61B 2018/00357; A61B 2018/00577; A61B 2018/00642; A61B 2018/00839

(57) ABSTRACT

A medical system may be configured to determine, at least in response to a selection or activation of a first transducer set, a second transducer set satisfying a determined positional relationship with respect to the first transducer set, each transducer in the second transducer set configured to record a respective electrogram, and the second transducer set mutually exclusive with the first transducer set. The medical system may be configured to visually display or increase a visual prominence of a portion of an electrogram set derived from or corresponding to the determined second transducer set.

### CROSS-REFERENCE TO RELATED APPLICATION

This application claims the benefit of U.S. Provisional Application No. 63/519,731, filed Aug. 15, 2023, the entire disclosure of which is hereby incorporated herein by reference.

### TECHNICAL FIELD

Aspects of this disclosure generally are related to systems and methods for electrogram set determination, such systems and methods applicable to medical systems.

### BACKGROUND

Cardiac surgery was initially undertaken using highly invasive open procedures. A sternotomy, which is a type of incision in the center of the chest that separates the sternum was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with various methods including a technique known as the “PV (pulmonary vein) isolation”. Research has shown that atrial fibrillation typically begins in the pulmonary veins or at the point where they attach to the left atrium. There are typically four major pulmonary veins, and some or all may be a focal point for activity that may cause atrial fibrillation. During this procedure, physicians create specific patterns of lesions in the heart to block various paths taken by the spurious electrical signals. The patterns of lesions may include a pattern of one or more lesions that encircle at least one of the pulmonary veins. Lesions were originally created using incisions, but are now typically created by ablating the tissue with various techniques including pulsed field ablation (PFA) (also known as irreversible electroporation), radiofrequency (“RF”) ablation, microwave ablation, laser ablation, and cryogenic ablation. Lesion formation may be performed with a high success rate under the direct vision that is provided in open procedures, but is relatively complex to perform intravascularly or percutaneously because of the difficulty in creating the lesions in the correct locations. The continuity, transmurality, and placement of the lesion patterns that are formed can impact the ability to block paths taken within the heart by spurious electrical signals. In this regard, the monitoring of electrograms (i.e., cardiac signals measured intracorporeally) may be used to determine the efficacy of the lesion patterns. Because of the complexity of percutaneous procedures and the increasing complexity of percutaneous devices that treat atrial fibrillation, including the increasing number of transducers of such devices, the amount of information and electrograms presented to a user or physician during a procedure can be excessive. In this regard, because timeliness and precision are important requirements for the treatment of atrial fibrillation, it can be time-consuming and can increase the risk of misunderstanding for a user or physician to have to visually sift through many electrograms and other real-time treatment data during a procedure. Accordingly, the present inventors recognized that there is a need in the art for improved intra-bodily-cavity transducer-based device systems or control mechanisms thereof with improved capabilities to more efficiently present relevant information about a treatment procedure.

### SUMMARY

At least the above-discussed need is addressed, and technical solutions are achieved by various embodiments of the present invention. According to some embodiments, a medical system may be summarized as including a data processing device system, an input-output device system communicatively connected to the data processing device system, and a memory device system communicatively connected to the data processing device system and storing a program executable by the data processing device system. According to various embodiments, the data processing device system may be configured by the program at least to receive input via the input-output device system indicating a selection of a first transducer set of a plurality of transducers of at least a portion of a transducer-based device, the at least the portion of the transducer-based device positionable within a bodily cavity, each transducer in the first transducer set configured to transmit tissue ablative energy. According to various embodiments, the data processing device system may be configured by the program at least to determine, at least in response to the selection of the first transducer set, a second transducer set of the plurality of transducers satisfying a determined positional relationship with respect to the first transducer set, each transducer in the second transducer set configured to record a respective electrogram, and the second transducer set mutually exclusive with the first transducer set. According to various embodiments, the data processing device system may be configured by the program at least to cause, via the input-output device system, display of at least part of an electrogram set. According to various embodiments, the data processing device system may be configured by the program at least to cause, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

In some embodiments, each transducer in the second transducer set may be configured to transmit tissue ablative energy. In some embodiments, each transducer in the first transducer set may be configured to record a respective electrogram. In some embodiments, each transducer in the first transducer set and each transducer in the second transducer set may include an electrode. In some embodiments, each transducer in the first transducer set may be configured to transmit particular energy configured to cause pulsed field ablation. In some embodiments, each transducer in the first transducer set may be configured to transmit particular energy configured to cause thermal ablation.

According to some embodiments, the positional relationship with respect to the first transducer set may be determined prior to the selection of the first transducer set. In some embodiments, the positional relationship with respect to the first transducer set may be determined based at least on particular data stored in the memory device system, the particular data indicating a predetermined positional relationship with respect to at least a selected transducer set. In some embodiments, the first transducer set may include at least three transducers of the plurality of transducers, the at least three transducers arranged at non-colinear locations bounding a region of space interior the non-colinear locations of the at least three transducers, and the data processing device system may be configured by the program at least to determine, for inclusion in the second transducer set, particular transducers of the plurality of transducers located at least in part or entirely in the region of space as satisfying the determined positional relationship with respect to the first transducer set. A location of each transducer in the first transducer set is arranged at a respective location, and the respective locations of at least some of the transducers in the first transducer set may bound the region of space. In some embodiments, various transducers in the first transducer set may be distributed to at least partially surround a particular anatomical feature in the bodily cavity. In some embodiments, the bodily cavity may be a cardiac cavity, and the particular anatomical feature is a bodily opening in the cardiac cavity.

In some embodiments, the first transducer set includes at least three transducers of the plurality of transducers. According to some embodiments, the at least three transducers may be arranged at non-colinear locations bounding a region of space exterior the non-colinear locations of the at least three transducers, and the data processing device system may be configured by the program at least to determine, for inclusion in the second transducer set, particular transducers of the plurality of transducers located at least in part or entirely in the region of space as satisfying the determined positional relationship with respect to the first transducer set. In some embodiments, the location of each transducer in the first transducer set is arranged at a respective location, and the respective locations of the transducers in the first transducer set may bound the region of space. In some embodiments, various transducers in the first transducer set may be distributed to at least partially surround a particular anatomical feature in the bodily cavity. In some embodiments, the bodily cavity may be a cardiac cavity, and the particular anatomical feature is a bodily opening in the cardiac cavity.

In some embodiments, the first transducer set may include at least two transducers of the plurality of transducers, the at least two transducers arranged at non-coincident locations intersected by a virtual plane. In some embodiments, the data processing device system may be configured by the program at least to determine, for inclusion in the second transducer set, particular transducers of the plurality of transducers as located at least in part or entirely on a first side of the virtual plane as satisfying the determined positional relationship with respect to the first transducer set. In some embodiments, the data processing device system may be configured by the program at least to determine the first side, a second side of the virtual plane, or each of the first side and the second side based at least on user input received via the input-output device system. In some embodiments, the data processing device system may be configured by the program at least to determine the first side, a second side of the virtual plane, or each of the first side and the second side based at least on an analysis of transducer data. In some embodiments, the transducer data may be provided by at least some of the plurality of transducers. In some embodiments, the data processing device system may be configured by the program at least to determine, via an analysis of transducer data, a propagation of electrophysiological activity through the virtual plane. In some embodiments, the data processing device system may be further configured by the program at least to determine the first side as a downstream side of the virtual plane, the downstream side of the virtual plane determined in accordance with a direction of the propagation of the electrophysiological activity through the virtual plane. In some embodiments, the transducer data may be provided by at least some of the plurality of transducers. In some embodiments, the data processing device system may be further configured by the program at least to determine the first side as an upstream side of the virtual plane, the upstream side of the virtual plane determined in accordance with a direction of the propagation of the electrophysiological activity through the virtual plane. In some embodiments, the transducer data may be provided by at least some of the plurality of transducers.

In some embodiments, the received input may include user-based input indicating the selection of the first transducer set. In some embodiments, the received input may include machine-based input indicating the selection of the first transducer set.

In some embodiments, (c) at least one electrogram in the first portion of the electrogram set is a unipolar electrogram, (d) at least one electrogram in the second portion of the electrogram set is a unipolar electrogram, or (c) and (d).

In some embodiments, (c) at least one electrogram in the first portion of the electrogram set is a bipolar electrogram, the bipolar electrogram derived from two electrograms recorded by two respective transducers in the second transducer set, (d) at least one electrogram in the second portion of the electrogram set is a bipolar electrogram, the bipolar electrogram derived from two electrograms recorded by two respective transducers in the second transducer set, or (c) and (d).

In some embodiments, (c) at least one electrogram in the first portion of the electrogram set is an omnipolar electrogram, the omnipolar electrogram derived from multiple electrograms recorded by multiple respective transducers in the second transducer set, (d) at least one electrogram in the second portion of the electrogram set is an omnipolar electrogram, the omnipolar electrogram derived from multiple electrograms recorded by multiple respective transducers in the second transducer set, or (c) and (d).

In some embodiments, the data processing device system may be configured by the program at least to cause, via the input-output device system, a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set. According to some embodiments, the data processing device system may be configured by the program at least to remove visual display, via the input-output device system and in response to at least the visual display of the first portion of the electrogram set in the state in which the first portion of the electrogram set was not visually displayed just prior to the visual display of the first portion of the electrogram set, of at least one electrogram in the third portion of the electrogram set that was displayed just prior to the visual display of the first portion of the electrogram set. In some embodiments, at least one transducer in the first transducer set may be configured to record an electrogram, and the third transducer set may include the at least one transducer in the first transducer set.

In some embodiments, the data processing device system may be configured by the program at least to cause, via the input-output device system, a visual display of the electrogram set in an ordered array of electrograms, and a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set. In some embodiments, the data processing device system may be configured by the program at least to cause, via the input-output device system, visual repositioning, in response to at least the visual display of the first portion of the electrogram set in the state in which the first portion of the electrogram set was not visually displayed just prior to the visual display of the first portion of the electrogram set, of at least some of the electrograms in the third portion of the electrogram set in the ordered array of electrograms. In some embodiments, at least one transducer in the first transducer set may be configured to record an electrogram, and wherein the third transducer set may include the at least one transducer in the first transducer set.

In some embodiments, the data processing device system may be configured by the program at least to cause, via the input-output device system, a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set. In some embodiments, the data processing device system may be configured by the program at least to cause, via the input-output device system, visual display, in response to at least the visual display of the first portion of the electrogram set in the state in which the first portion of the electrogram set was not visually displayed just prior to the visual display of the first portion of the electrogram set, of each electrogram of the first portion of the electrogram set with a greater visual prominence than the visual display of each of at least some of the electrograms in the third portion of the electrogram set. In some embodiments, at least one transducer in the first transducer set may be configured to record an electrogram, and wherein the at least some of the transducers in the third transducer set may include the at least one transducer in the first transducer set.

In some embodiments, the data processing device system may be configured by the program at least to cause, via the input-output device system, a visual display of the electrogram set in an ordered array of electrograms, at least some of the electrograms in the second portion of the electrogram set separated from one another in the ordered array of electrograms by one or more electrograms not forming part of the second portion of the electrogram set. In some embodiments, the data processing device system may be configured by the program at least to cause (b) at least by reordering the electrograms in the ordered array of electrograms to form a reordered array of electrograms such that all the electrograms of the second portion of the electrogram set are arranged successively one after the other in the reordered array of electrograms without any interruption by any electrogram in the electrogram set not forming part of the second portion of the electrogram set. In some embodiments, at least one transducer in the first transducer set may be configured to record an electrogram, and the one or more electrograms not forming part of the second portion of the electrogram set may include an electrogram recorded by the at least one transducer in the first transducer set. In some embodiments, the data processing device system may be configured by the program at least to cause (b) at least by causing, via the input-output device system, the reordering of the electrograms in the ordered array of electrograms to form the reordered array of electrograms such that all of the electrograms of the second portion of the electrogram set appear at a beginning of the reordered array of electrograms.

In some embodiments, the data processing device system may be configured by the program at least to cause (a) and (b) at least by causing a visual display, via the input-output device system, of the first portion of the electrogram set and the second portion of the electrogram set successively one after the other in an ordered array of electrograms without any interruption by any electrogram in the electrogram set not forming part of a group of electrograms consisting of the first portion of the electrogram set and the second portion of the electrogram set.

In some embodiments, the data processing device system may be configured by the program at least to cause (b) at least by causing, via the input-output device system, each electrogram of the second portion of the electrogram set to be displayed according to a first visual characteristic set just prior to the increasing of the visual prominence of the second portion of the electrogram set, and cause via the input-output device system, each electrogram of the second portion of the electrogram set to be displayed according to a second visual characteristic set upon the increasing of the visual prominence of the second portion of the electrogram set, the second visual characteristic set different than the first visual characteristic set. In some embodiments, the data processing device system may be configured by the program at least to cause (a) at least by causing, via the input-output device system, each electrogram of the first portion of the electrogram set to be displayed according to the second visual characteristic set. In some embodiments, each electrogram of the second portion of the electrogram set is displayed as a signal trace, and each signal trace may be displayed with (i) a first graphical line type according to the first visual characteristic set, and (ii) a second graphical line type according to the second visual characteristic set, the second graphical line type more visually prominent than the first graphical line type.

In some embodiments, the data processing device system may be configured by the program at least to cause, via the input-output device system, a visual display of a third portion of the electrogram set just prior to the increasing of the visual prominence of the second portion of the electrogram set. According to some embodiments, no electrogram in the third portion of the electrogram set is provided by any electrogram in the second portion of the electrogram set, and each electrogram of the third portion of the electrogram set may be displayed according to a first visual characteristic set just prior to the increasing of the visual prominence of the second portion of the electrogram set. In some embodiments, each electrogram of the third portion of the electrogram set may be displayed according to a second visual characteristic set upon the increasing of the visual prominence of the second portion of the electrogram set, the second visual characteristic set less visually prominent than the first visual characteristic set. In some embodiments, each electrogram of the third portion of the electrogram set may be displayed with (i) a first degree of opacity according to the first visual characteristic set, and (ii) a second degree of opacity according to the second visual characteristic set, the second degree of opacity being less that the first degree of opacity. In some embodiments, at least one transducer in the first transducer set may be configured to record an electrogram, and the third portion of the electrogram set may include an electrogram recorded by the at least one transducer in the first transducer set.

In some embodiments, the data processing device system may be configured by the program at least to cause, via the input-output device system, (a), (b), or (a) and (b) at least in response to the determination of the second transducer set.

According to some embodiments, a medical system may be summarized as including a data processing device system, an input-output device system communicatively connected to the data processing device system, and a memory device system communicatively connected to the data processing device system and storing a program executable by the data processing device system. According to various embodiments, the data processing device system may be configured by the program at least to cause activation, via the input-output device system, of a first transducer set of a plurality of transducers of at least a portion of a transducer-based device, the at least the portion of the transducer-based device positionable within a bodily cavity, and the activation of the first transducer set causing each transducer of the first transducer set to transmit tissue ablative energy. According to various embodiments, the data processing device system may be configured by the program at least to determine, at least in response to the activation of the first transducer set, a second transducer set of the plurality of transducers satisfying a determined positional relationship with respect to the first transducer set, each transducer in the second transducer set configured to record a respective electrogram, and the second transducer set mutually exclusive with the first transducer set. According to various embodiments, the data processing device system may be configured by the program at least to cause, via the input-output device system, display of at least part of an electrogram set. According to various embodiments, the data processing device system may be configured by the program at least to cause, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

In some embodiments, each transducer in the second transducer set may be configured to transmit tissue ablative energy. In some embodiments, each transducer in the first transducer set may be configured to record a respective electrogram. In some embodiments, the data processing device system may be configured by the program at least to cause (a), (b), or (a) and (b) at least in response to initiation of activation of at least one transducer in the first transducer set. In some embodiments, the data processing device system may be configured by the program at least to cause (a), (b), or (a) and (b) at least in response to completion of activation of at least one transducer in the first transducer set. In some embodiments, each transducer in the first transducer set may be configured to transmit particular energy configured to cause pulsed field ablation. In some embodiments, each transducer in the first transducer set may be configured to transmit particular energy configured to cause thermal ablation.

In some embodiments, the positional relationship with respect to the first transducer set may be determined prior to the activation of the first transducer set. In some embodiments, the positional relationship with respect to the first transducer set may be determined based at least on particular data stored in the memory device system, the particular data indicating a predetermined positional relationship with respect to at least an activated transducer set. In some embodiments, the first transducer set may include at least three transducers of the plurality of transducers, the at least three transducers arranged at non-colinear locations bounding a region of space interior the non-colinear locations of the at least three transducers, and the data processing device system may be configured by the program at least to determine, for inclusion in the second transducer set, particular transducers of the plurality of transducers located at least in part or entirely in the region of space as satisfying the determined positional relationship with respect to the first transducer set. In some embodiments, a location of each transducer in the first transducer set may be arranged at a respective location, the respective locations of at least some of the transducers in the first transducer set bounding the region of space. In some embodiments, various transducers in the first transducer set may be distributed to at least partially surround a particular anatomical feature in the bodily cavity. In some embodiments, the bodily cavity may be a cardiac cavity, and the particular anatomical feature may be a bodily opening in the cardiac cavity.

In some embodiments, the first transducer set may include at least three transducers of the plurality of transducers, the at least three transducers arranged at non-colincar locations bounding a region of space exterior the non-colinear locations of the at least three transducers, and the data processing device system may be configured by the program at least to determine, for inclusion in the second transducer set, particular transducers of the plurality of transducers located at least in part or entirely in the region of space as satisfying the determined positional relationship with respect to the first transducer set. In some embodiments, the location of each transducer in the first transducer set may be arranged at a respective location, the respective locations of the transducers in the first transducer set bounding the region of space. In some embodiments, various transducers in the first transducer set may be distributed to at least partially surround a particular anatomical feature in the bodily cavity. In some embodiments, the bodily cavity may be a cardiac cavity, and the particular anatomical feature may be a bodily opening in the cardiac cavity.

In some embodiments, the first transducer set may include at least two transducers of the plurality of transducers, the at least two transducers arranged at non-coincident locations intersected by a virtual plane, and the data processing device system may be configured by the program at least to determine, for inclusion in the second transducer set, particular transducers of the plurality of transducers as located at least in part or entirely on a first side of the virtual plane as satisfying the determined positional relationship with respect to the first transducer set.

In some embodiments, the data processing device system may be configured by the program at least to determine the first side, a second side of the virtual plane, or each of the first side and the second side based at least on user input received via the input-output device system. In some embodiments, the data processing device system may be configured by the program at least to determine the first side, a second side of the virtual plane, or each of the first side and the second side based at least on an analysis of transducer data. In some embodiments, the transducer data may be provided by at least some of the plurality of transducers. In some embodiments, the data processing device system may be configured by the program at least to determine, via an analysis of transducer data, a propagation of electrophysiological activity through the virtual plane, the data processing device system may be further configured by the program at least to determine the first side as a downstream side of the virtual plane, the downstream side of the virtual plane determined in accordance with a direction of the propagation of the electrophysiological activity through the virtual plane. In some embodiments, the transducer data may be provided by at least some of the plurality of transducers. In some embodiments, the data processing device system may be configured by the program at least to determine, via an analysis of transducer data, a propagation of electrophysiological activity through the virtual plane, the data processing device system may be further configured by the program at least to determine the first side as an upstream side of the virtual plane, the upstream side of the virtual plane determined in accordance with a direction of the propagation of the electrophysiological activity through the virtual plane. In some embodiments, the transducer data may be provided by at least some of the plurality of transducers.

In some embodiments, (c) at least one electrogram in the first portion of the electrogram set may be a unipolar electrogram, (d) at least one electrogram in the second portion of the electrogram set may be a unipolar electrogram, or (c) and (d).

In some embodiments, (c) at least one electrogram in the first portion of the electrogram set may be a bipolar electrogram, the bipolar electrogram derived from two electrograms recorded by two respective transducers in the second transducer set, (d) at least one electrogram in the second portion of the electrogram set may be a bipolar electrogram, the bipolar electrogram derived from two electrograms recorded by two respective transducers in the second transducer set, or (c) and (d).

In some embodiments, (c) at least one electrogram in the first portion of the electrogram set may be an omnipolar electrogram, the omnipolar electrogram derived from multiple electrograms recorded by multiple respective transducers in the second transducer set, (d) at least one electrogram in the second portion of the electrogram set may be an omnipolar electrogram, the omnipolar electrogram derived from multiple electrograms recorded by multiple respective transducers in the second transducer set, or (c) and (d).

In some embodiments, the data processing device system may be configured by the program at least to cause, via the input-output device system, a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set, and the data processing device system may be configured by the program at least to remove visual display, via the input-output device system and in response to at least the visual display of the first portion of the electrogram set in the state in which the first portion of the electrogram set was not visually displayed just prior to the visual display of the first portion of the electrogram set, of at least one electrogram in the third portion of the electrogram set that was displayed just prior to the visual display of the first portion of the electrogram set. In some embodiments, at least one transducer in the first transducer set may be configured to record an electrogram, and the third transducer set may include the at least one transducer in the first transducer set.

In some embodiments, the data processing device system may be configured by the program at least to cause, via the input-output device system: a visual display of the electrogram set in an ordered array of electrograms; a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set; and visual repositioning, in response to at least the visual display of the first portion of the electrogram set in the state in which the first portion of the electrogram set was not visually displayed just prior to the visual display of the first portion of the electrogram set, of at least some of the electrograms in the third portion of the electrogram set in the ordered array of electrograms. In some embodiments, at least one transducer in the first transducer set is configured to record an electrogram, and the third transducer set may include the at least one transducer in the first transducer set.

In some embodiments, the data processing device system may be configured by the program at least to cause, via the input-output device system: a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set; and visual display, in response to at least the visual display of the first portion of the electrogram set in the state in which the first portion of the electrogram set was not visually displayed just prior to the visual display of the first portion of the electrogram set, of each electrogram of the first portion of the electrogram set with a greater visual prominence than the visual display of each of at least some of the electrograms in the third portion of the electrogram set. In some embodiments, at least one transducer in the first transducer set may be configured to record an electrogram, and the at least some of the transducers in the third transducer set may include the at least one transducer in the first transducer set.

In some embodiments, the data processing device system may be configured by the program at least to cause, via the input-output device system, a visual display of the electrogram set in an ordered array of electrograms, at least some of the electrograms in the second portion of the electrogram set separated from one another in the ordered array of electrograms by one or more electrograms not forming part of the second portion of the electrogram set. In some embodiments, the data processing device system may be configured by the program at least to cause (b) at least by reordering the electrograms in the ordered array of electrograms to form a reordered array of electrograms such that all the electrograms of the second portion of the electrogram set are arranged successively one after the other in the reordered array of electrograms without any interruption by any electrogram in the electrogram set not forming part of the second portion of the electrogram set. In some embodiments, at least one transducer in the first transducer set may be configured to record an electrogram, and the one or more electrograms not forming part of the second portion of the electrogram set may include an electrogram recorded by the at least one transducer in the first transducer set. In some embodiments, the data processing device system may be configured by the program at least to cause (b) at least by causing, via the input-output device system, the reordering of the electrograms in the ordered array of electrograms to form the reordered array of electrograms such that all of the electrograms of the second portion of the electrogram set appear at a beginning of the reordered array of electrograms.

In some embodiments, the data processing device system may be configured by the program at least to cause (a) and (b) at least by causing a visual display, via the input-output device system, of the first portion of the electrogram set and the second portion of the electrogram set successively one after the other in an ordered array of electrograms without any interruption by any electrogram in the electrogram set not forming part of a group of electrograms consisting of the first portion of the electrogram set and the second portion of the electrogram set.

In some embodiments, the data processing device system may be configured by the program at least to cause (b) at least by causing, via the input-output device system, each electrogram of the second portion of the electrogram set to be displayed according to a first visual characteristic set just prior to the increasing of the visual prominence of the second portion of the electrogram set, and cause via the input-output device system, each electrogram of the second portion of the electrogram set to be displayed according to a second visual characteristic set upon the increasing of the visual prominence of the second portion of the electrogram set, the second visual characteristic set different than the first visual characteristic set. In some embodiments, the data processing device system may be configured by the program at least to cause (a) at least by causing, via the input-output device system, each electrogram of the first portion of the electrogram set to be displayed according to the second visual characteristic set. In some embodiments, each electrogram of the second portion of the electrogram set may be displayed as a signal trace, each signal trace displayed with (i) a first graphical line type according to the first visual characteristic set, and (ii) a second graphical line type according to the second visual characteristic set, the second graphical line type more visually prominent than the first graphical line type.

In some embodiments, the data processing device system may be configured by the program at least to cause, via the input-output device system, a visual display of a third portion of the electrogram set just prior to the increasing of the visual prominence of the second portion of the electrogram set. According to some embodiments, no electrogram in the third portion of the electrogram set is provided by any electrogram in the second portion of the electrogram set, each electrogram of the third portion of the electrogram set may be displayed according to a first visual characteristic set just prior to the increasing of the visual prominence of the second portion of the electrogram set. In some embodiments, each electrogram of the third portion of the electrogram set may be displayed according to a second visual characteristic set upon the increasing of the visual prominence of the second portion of the electrogram set, the second visual characteristic set less visually prominent than the first visual characteristic set. In some embodiments, each electrogram of the third portion of the electrogram set may be displayed with (i) a first degree of opacity according to the first visual characteristic set, and (ii) a second degree of opacity according to the second visual characteristic set, the second degree of opacity being less that the first degree of opacity. In some embodiments, at least one transducer in the first transducer set may be configured to record an electrogram, and the third portion of the electrogram set may include an electrogram recorded by the at least one transducer in the first transducer set.

In some embodiments, the data processing device system may be configured by the program at least to cause, via the input-output device system, (a), (b), or (a) and (b) at least in response to the determination of the second transducer set.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

For example, in some embodiments, a method may be executed by a data processing device system according to a program stored by a communicatively connected memory device system, the data processing device system also communicatively connected to an input-output device system, and the method may be summarized as including: receiving input via the input-output device system indicating a selection of a first transducer set of a plurality of transducers of at least a portion of a transducer-based device, the at least the portion of the transducer-based device positionable within a bodily cavity, each transducer in the first transducer set configured to transmit tissue ablative energy; determining, at least in response to the selection of the first transducer set, a second transducer set of the plurality of transducers satisfying a determined positional relationship with respect to the first transducer set, each transducer in the second transducer set configured to record a respective electrogram, and the second transducer set mutually exclusive with the first transducer set; causing, via the input-output device system, display of at least part of an electrogram set; and causing, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

For another example, in some embodiments, a method may be executed by a data processing device system according to a program stored by a communicatively connected memory device system, the data processing device system also communicatively connected to an input-output device system, and the method may be summarized as including: causing activation, via the input-output device system, of a first transducer set of a plurality of transducers of at least a portion of a transducer-based device, the at least the portion of the transducer-based device positionable within a bodily cavity, and the activation of the first transducer set causing each transducer of the first transducer set to transmit tissue ablative energy; determining, at least in response to the activation of the first transducer set, a second transducer set of the plurality of transducers satisfying a determined positional relationship with respect to the first transducer set, each transducer in the second transducer set configured to record a respective electrogram, and the second transducer set mutually exclusive with the first transducer set; causing, via the input-output device system, display of at least part of an electrogram set; and causing, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

For example, in some embodiments, one or more computer-readable storage mediums may be provided storing a program executable by a data processing device system communicatively connected to an input-output device system, and the program may be summarized as including: reception instructions configured to cause reception of input via the input-output device system indicating a selection of a first transducer set of a plurality of transducers of at least a portion of a transducer-based device, the at least the portion of the transducer-based device positionable within a bodily cavity, each transducer in the first transducer set configured to transmit tissue ablative energy; determination instructions configured to cause determination, at least in response to the selection of the first transducer set, of a second transducer set of the plurality of transducers satisfying a determined positional relationship with respect to the first transducer set, each transducer in the second transducer set configured to record a respective electrogram, and the second transducer set mutually exclusive with the first transducer set; first display instructions configured to cause, via the input-output device system, display of at least part of an electrogram set; and second display instructions configured to cause, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

For another example, in some embodiments, one or more computer-readable storage mediums may be provided storing a program executable by a data processing device system communicatively connected to an input-output device system, and the program may be summarized as including: activation instructions configured to cause activation, via the input-output device system, of a first transducer set of a plurality of transducers of at least a portion of a transducer-based device, the at least the portion of the transducer-based device positionable within a bodily cavity, and the activation of the first transducer set causing each transducer of the first transducer set to transmit tissue ablative energy; determination instructions configured to cause determination, at least in response to the activation of the first transducer set, of a second transducer set of the plurality of transducers satisfying a determined positional relationship with respect to the first transducer set, each transducer in the second transducer set configured to record a respective electrogram, and the second transducer set mutually exclusive with the first transducer set; first display instructions configured to cause, via the input-output device system, display of at least part of an electrogram set; and second display instructions configured to cause, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

In some embodiments, each of any of one or more or all of the computer-readable storage mediums or medium systems (also referred to as processor-accessible memory device systems) described herein is a non-transitory computer-readable (or processor-accessible) storage medium or medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 includes a schematic representation of a transducer-activation system according to various example embodiments, the transducer-activation system including a data processing device system, an input-output device system, and a memory device system.

FIG. 2 includes a cutaway diagram of a heart showing a transducer-based device percutaneously placed in a left atrium of the heart, according to various example embodiments.

FIG. 3A includes a partially schematic representation of a medical system according to various example embodiments, the medical system including a data processing device system, an input-output device system, a memory device system, and a transducer-based device including a plurality of transducers and an expandable structure shown in a delivery or unexpanded configuration.

FIG. 3B includes the representation of the medical system of FIG. 3A with the expandable structure shown in a deployed or expanded configuration, according to some embodiments.

FIG. 4 includes a schematic representation of a transducer-based device that includes a flexible circuit structure, according to various example embodiments.

FIGS. 5A and 5B include block diagrams of various methods for electrogram determination, according to some embodiments.

FIGS. 6A-6E illustrate various graphical representations of transducers and electrograms, according to various embodiments.

FIG. 7 illustrates a virtual plane intersecting a transducer set, according to some embodiments.

### DETAILED DESCRIPTION

The above-discussed need in the art is addressed and technical solutions are achieved according to various embodiments of the present invention. In some embodiments, an efficient presentation of electrograms is provided. For instance, in some embodiments, electrograms from transducers other than or in addition to transducers selected or activated for ablation are visually presented or their visual prominence is increased to assist a user or physician with seeing electrograms that help indicate the efficacy of an ablation procedure (e.g., whether a successful conduction block configured to block electrophysiological activity has occurred, in some embodiments). For example, the display of electrograms detected by a particular set of transducers inside of a ringed arrangement of transducers selected to transmit ablative energy can be useful for assessing the presence of electrical isolation in the lesion formed by the ringed arrangement of transducers. On the other hand, the display of electrograms detected by a particular set of transducers that are outside of a ringed arrangement of transducers selected to transmit ablative energy may be useful as a positive control for artificial stimulation (e.g., a pacing signal emitted by a transducer) and for assessing the delay of any observed entry into the lesion formed by the ringed arrangement of transducers, according to some embodiments.

In some embodiments, a selection of a first transducer set of a plurality of transducers of at least a portion of a transducer-based device is made. According to various embodiments, the at least the portion of the transducer-based device is positionable within a bodily cavity, and each transducer in the first transducer set is activatable to transmit tissue ablative energy. In some embodiments, a machine-based determination of a second transducer set of the plurality of transducers is made at least in response to the selection of the first transducer set or at least in response to the activation of the first transducer set. In some embodiments, each transducer in the second transducer set is configured to record a respective electrogram and the second transducer set is mutually exclusive with the first transducer set. In some embodiments, at least part of an electrogram set is displayed, and (a) a visual display of a first portion of the electrogram set is caused in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set is caused in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

In some embodiments, the transducers selected as part of the first transducer set may be arranged along an encircling path, and the second transducer set may be selected as one or more particular ones of the plurality of transducers that satisfy a determined positional relationship in which one or more transducers selected as part of the second transducer set is or are located inside of the encircling path. In some embodiments, the transducers selected as part of the first transducer set may be arranged along an encircling path, and the second transducer set may be selected as one or more particular ones of the plurality of transducers that satisfy a determined positional relationship in which one or more transducers selected as part of the second transducer set is or are located outside of the encircling path.

Such an architecture, according to various embodiments, allows, among other possibilities and benefits, a user (e.g., health care practitioner) to more easily identify particular electrograms, and in some embodiments, their respective corresponding transducers in a myriad of electrograms that are displayed or can be displayed. In this regard, the user can make a more informed decision more quickly of the effectiveness of the first transducer set when activated. This architecture allows at least for a particularly efficient workflow when a machine or data processing device system automatically selects the second transducer set (e.g., inside or outside of the encircling path in some embodiments).

In the following description, certain specific details are set forth in order to provide a thorough understanding of various embodiments of the invention. However, one skilled in the art will understand that the invention may be practiced at a more general level without these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily always referring to one embodiment or a same embodiment. Furthermore, the particular features, structures, or characteristics of different embodiments may be combined in any suitable manner to form one or more other embodiments. In one embodiment, all references to “some embodiments” may refer to the same single embodiment.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A. The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments, and the word “system” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers or is a necessary precondition for the event A, according to various embodiments.

The phrase “thermal ablation” as used in this disclosure refers, in some embodiments, to an ablation method in which destruction of tissue occurs by hyperthermia (elevated tissue temperatures) or hypothermia (depressed tissue temperatures). Thermal ablation may include RF ablation, microwave ablation, or cryo-ablation by way of non-limiting example. RF ablation energy waveforms can take various forms. For example, in some RF ablation embodiments, energy is provided in the form of a continuous waveform. In some RF ablation embodiments, energy is provided in the form of discrete energy applications (e.g., in the form of a duty-cycled waveform).

The phrase “pulsed field ablation” (“PFA”) as used in this disclosure refers, in some embodiments, to an ablation method that employs high voltage pulse delivery in a unipolar or bipolar fashion in proximity to target tissue. In some embodiments, each high voltage pulse may be referred to as a discrete energy application. In some embodiments, a grouped plurality of high voltages pulses may be referred to as a discrete energy application. Each high voltage pulse can be a monophasic pulse including a single polarity, or a biphasic pulse including a first component having a first particular polarity and a second component having a second particular polarity opposite the first particular polarity. In some embodiments, the second component of the biphasic pulse follows immediately after the first component of the biphasic pulse. In some embodiments, the first and second components of the biphasic pulse are temporally separated by a relatively small time interval. In some embodiments, each high voltage pulse may include a multiphasic pulse, such as a triphasic pulse, that includes a first component having a first particular polarity, a second component having a second particular polarity opposite the first particular polarity, and a third component having a third particular polarity that is the same as the first particular polarity. The electric field applied by the high voltage pulses in PFA physiologically changes the tissue cells to which the energy is applied (e.g., puncturing or perforating the cell membrane to form various pores therein). If a lower field strength is established, the formed pores may close in time and cause the cells to maintain viability (e.g., a process sometimes referred to as reversible electroporation). If the field strength that is established is greater, then permanent, and sometimes larger, pores form in the tissue cells, the pores allowing loss of control of ion concentration gradients (both inward and outward) thereby resulting in cell death (e.g., a process sometimes referred to as irreversible electroporation).

According to some embodiments, the word “fluid” as used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intracardiac cavities (e.g., a left atrium or a right atrium).

According to some embodiments, the words “bodily opening” as used in this disclosure should be understood to include a naturally occurring bodily opening or channel or lumen; a bodily opening or channel or lumen formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens, or channels and positioned within the bodily opening (e.g., a catheter sheath) may be present in various embodiments. These elements may provide a passageway through a bodily opening for various devices employed in various embodiments.

The words “bodily cavity” as used in this disclosure should be understood to mean a cavity in a body, in some embodiments. The bodily cavity may be a cavity or chamber provided in a bodily organ (e.g., an intracardiac cavity of a heart).

The word “tissue” as used in some embodiments in this disclosure should be understood to include any surface-forming tissue that is used to form a surface of a body or a surface within a bodily cavity, a surface of an anatomical feature or a surface of a feature associated with a bodily opening positioned in fluid communication with the bodily cavity. The tissue can include part, or all, of a tissue wall or membrane that defines a surface of the bodily cavity. In this regard, the tissue can form an interior surface of the cavity that surrounds a fluid within the cavity. In the case of cardiac applications, tissue can include tissue used to form an interior surface of an intracardiac cavity such as a left atrium or a right atrium. In some embodiments, the word tissue can refer to a tissue having fluidic properties (e.g., blood) and may be referred to as fluidic tissue.

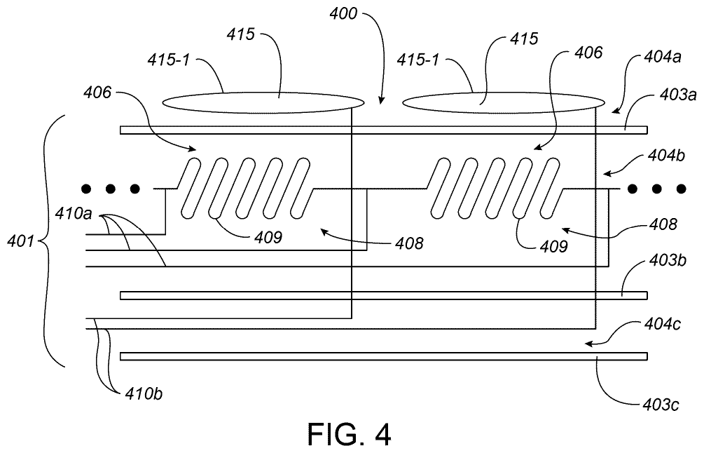

According to some embodiment, the term “transducer” as used in this disclosure should be interpreted broadly as any device capable of transmitting or delivering energy, distinguishing between fluid and tissue, sensing temperature, creating heat, ablating tissue, sensing, sampling or measuring electrical activity of a tissue surface (e.g., sensing, sampling or measuring intracardiac electrograms, or sensing, sampling or measuring intracardiac voltage data), stimulating tissue, providing location information (e.g., in conjunction with a navigation system), or any combination thereof. A transducer may convert input energy of one form into output energy of another form. Without limitation, a transducer may include an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., at least with FIG. 4 discussed below.

The term “activation” as used in this disclosure, according to some embodiments, should be interpreted broadly as making active a particular function as related to various transducers disclosed in this disclosure. Particular functions may include, but are not limited to, tissue ablation (e.g., PFA or thermal ablation such as RF), sensing, sampling, or measuring electrophysiological activity (e.g., sensing, sampling, or measuring intracardiac electrogram information, or sensing, sampling, or measuring intracardiac voltage data), sensing, sampling, or measuring temperature, and sensing, sampling, or measuring electrical characteristics (e.g., tissue impedance or tissue conductivity). For example, in some embodiments, activation of a tissue ablation function of a particular transducer is initiated by causing energy sufficient for tissue ablation from an energy source device system to be delivered to the particular transducer. Also, in this example, the activation can last for a duration of time concluding when the ablation function is no longer active, such as when energy sufficient for the tissue ablation is no longer provided to the particular transducer. In some contexts and embodiments, however, the word “activation” can merely refer to the initiation of the activating of a particular function, as opposed to referring to both the initiation of the activating of the particular function and the subsequent duration in which the particular function is active. In these contexts, the phrase or a phrase similar to “activation initiation” may be used.

The term “electrogram” as used in this disclosure refers to, in some embodiments, a display of information indicating the electrical potentials of a tissue (e.g., the heart) at localized positions within the body. Typically, electrograms are recorded by transducers (e.g., electrodes) placed directly within a body instead of on an exterior surface of the body. According to various embodiments, the recorded electrograms are graphically displayed in a graphical representation. For example, an electrogram may be displayed as a signal or tracing representing the electrical potentials within the body. In cardiac electrophysiological studies, various electrogram recordings (e.g., unipolar, bipolar, or omnipolar) are used to identify the sequence of activation of electrophysiological activity within the heart. Such recordings can lead to the identification of critical areas of an arrhythmia circuit which can then be the target for ablation.

The term “electrocardiogram” as used in this disclosure refers to, in some embodiments, a display of information providing an interpretation of the electrical activity of the heart over a time period. Electrocardiograms are detected by electrodes attached to an external or skin-based surface of the body. In this regard, unlike electrograms, electrocardiograms are generated transthoracically (i.e., across the thorax or chest).

In the following description, some embodiments of the present invention may be implemented at least in part by a data processing device system, or a controller system, configured by a software program. Such a program may equivalently be implemented as multiple programs, and some, or all, of such software program(s) may be equivalently constructed in hardware. In this regard, reference to “a program” should be interpreted to include one or more programs.

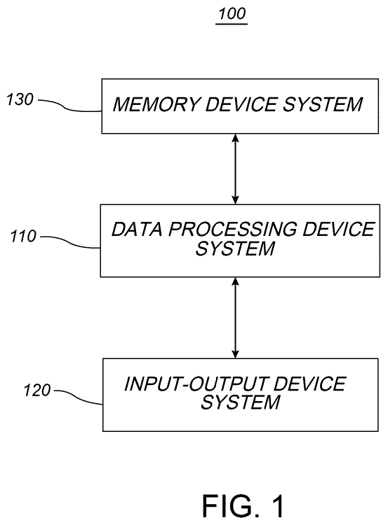

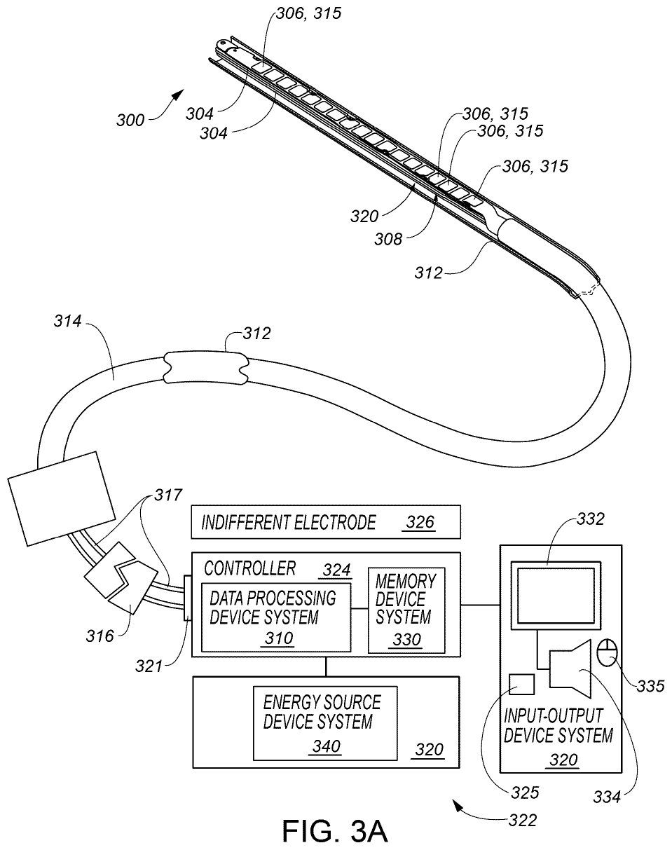

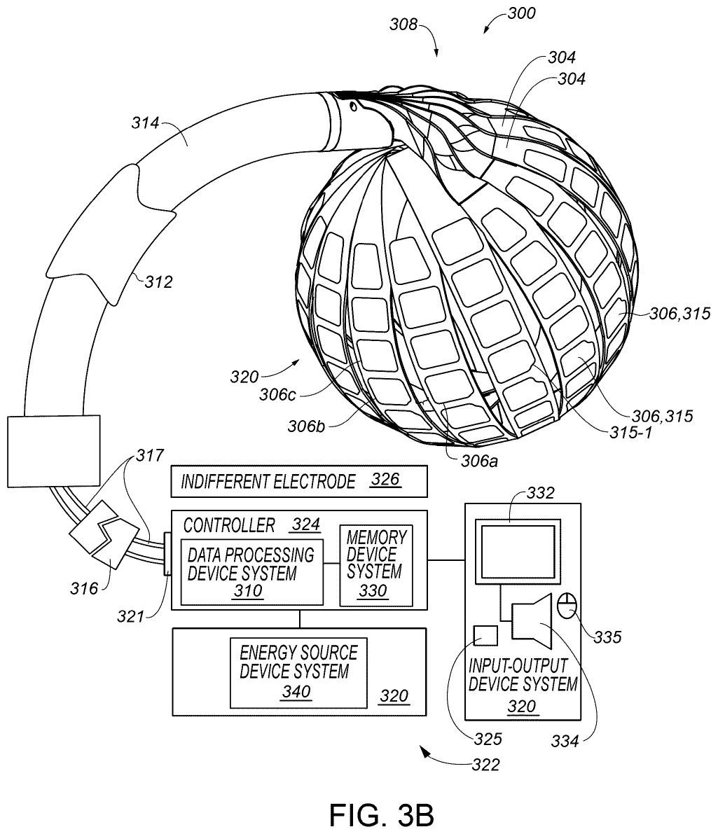

According to some embodiments, the term “program” in this disclosure should be interpreted to include one or more programs including a set of instructions or modules that can be executed by one or more components in a system, such as a controller system or a data processing device system, in order to cause the system to perform one or more operations. The set of instructions or modules may be stored by any kind of memory device, such as those described subsequently with respect to the memory device system 130 or 330 shown in at least FIGS. 1 and 3, respectively. In addition, this disclosure may describe or similarly describe that the instructions or modules of a program are configured to cause the performance of an action. The phrase “configured to” in this context is intended to include, for example, at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. In some instances, this disclosure describes that the instructions or modules of a program perform a function. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data or the like includes modifications to that information or data. For example, “data X” may be encrypted for transmission, and a reference to “data X” is intended to include both its encrypted and unencrypted forms, unless otherwise required or indicated by context. For another example, “image information Y” may undergo a noise filtering process, and a reference to “image information Y” is intended to include both the pre-processed form and the noise-filtered form, unless otherwise required or indicated by context. In other words, both the pre-processed form and the noise-filtered form are considered to be “image information Y”, unless otherwise required or indicated by context. In order to stress this point, the phrase “or a derivative thereof” or the like may be used herein. Continuing the preceding example, the phrase “image information Y or a derivative thereof” refers to both the pre-processed form and the noise-filtered form of “image information Y”, unless otherwise required or indicated by context, with the noise-filtered form potentially being considered a derivative of “image information Y”. However, non-usage of the phrase “or a derivative thereof” or the like nonetheless includes derivatives or modifications of information or data unless otherwise explicitly noted or required by context.

In some embodiments, the phrase “graphical representation” used herein is intended to include a visual representation presented via a display device system and may include computer-generated text, graphics, animations, or one or more combinations thereof, which may include one or more visual representations originally generated, at least in part, by an image-capture device, such as computerized tomography (“CT”) scan images, magnetic resonance imaging (“MRI”) images, or images created from a navigation system (e.g., electric potential navigation system or an electromagnetic navigation system), according to some embodiments. The graphical representation may include various entities depicted in a two-dimensional manner. The graphical representation may include various entities depicted in a three-dimensional manner, in some embodiments. The graphical representation may include other information, including recorded data such as electrograms.

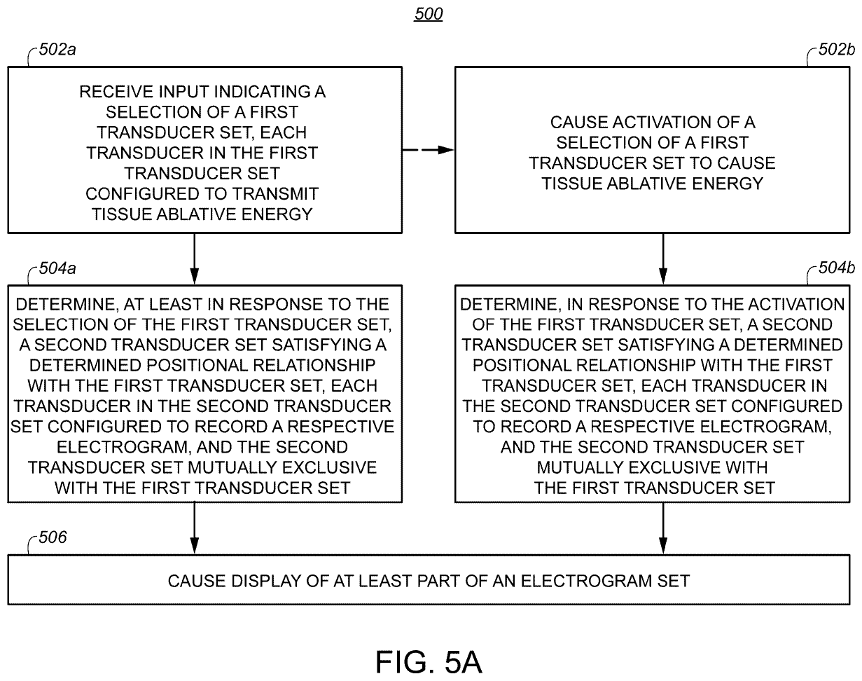

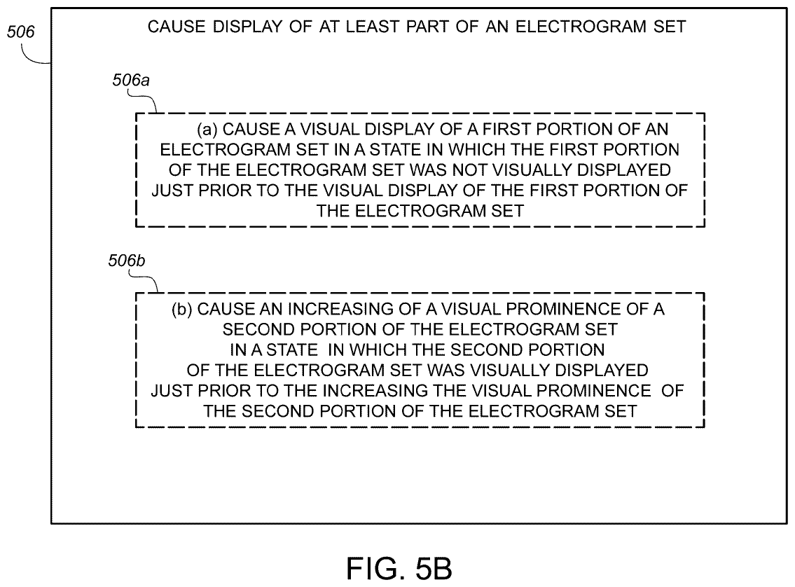

Example methods are described herein with respect to FIGS. 5A-5B. Such figures include blocks associated with actions, computer-executable instructions, or both, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in each of the method figures herein is not limited to an actual structure of any program or set of instructions or required ordering of method tasks, and such method figures, according to some embodiments, merely illustrate the tasks that instructions are configured to perform, for example, upon execution by a data processing device system in conjunction with interactions with one or more other devices or device systems.

Each of the phrases “derived from” or “derivation of” or “derivation thereof” or the like may be used herein, according to some embodiments, to mean to come from at least some part of a source, be created from at least some part of a source, or be developed as a result of a process in which at least some part of a source forms an input, according to various embodiments. For example, a data set derived from some particular portion of data may include at least some part of the particular portion of data, or may be created from at least part of the particular portion of data, or may be developed in response to a data manipulation process in which at least part of the particular portion of data forms an input. In some embodiments, a data set may be derived from a subset of the particular portion of data. In some embodiments, the particular portion of data is analyzed to identify a particular subset of the particular portion of data, and a data set is derived from the subset. In various ones of these embodiments, the subset may include some, but not all, of the particular portion of data. In some embodiments, changes in at least one part of a particular portion of data may result in changes in a data set derived at least in part from the particular portion of data.

In this regard, each of the phrases “derived from” or “derivation of” or “derivation thereof” or the like may be used herein merely to emphasize the possibility that such data or information may be modified or subject to one or more operations. For example, if a device generates first data for display, the process of converting the generated first data into a format capable of being displayed may alter the first data. This altered form of the first data may be considered a derivative or derivation of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative or derivation of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended in some embodiments to include these and like changes, regardless of whether or not the phrase “derived from” or “derivation of” or “derivation thereof” or the like is used in reference to the information or data.

As indicated above, usage of the phrase “derived from” or “derivation of” or “derivation thereof” or the like merely emphasizes the possibility of such changes. Accordingly, in some embodiments, the usage, non-usage, addition of, or deletion of the phrase “derived from” or “derivation of” or “derivation thereof” or the like should have no impact on the interpretation of the respective data or information. For example, the above-discussed color-coded bar chart may be considered a derivative of the respective first data or may be considered the respective first data itself, whether or not the phrase “derived from” or “derivation of” or “derivation thereof” or the like is used, according to some embodiments.

In some embodiments, the term “adjacent”, the term “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the term “adjacent”, the term “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In the context of graphical elements, discussed at various points in this description, two graphical elements may be considered adjacent, in some embodiments, when the two graphical elements have no other graphical element between them that is the same in form to any of the two graphical elements. In the context of graphical elements, discussed at various points in this disclosure, two graphical elements may be considered adjacent, in some embodiments, when the two graphical elements have no other graphical element between them that is the same in function to any of the two graphical elements. In the context of transducer graphical elements, discussed at various points in this disclosure, two transducer graphical elements may be considered adjacent, in some embodiments, when the two transducer graphical elements have no other transducer graphical element between them. In the context of graphical elements taking the form of electrograms, discussed at various points in this disclosure, two electrograms may be considered adjacent (e.g., in an ordered array), in some embodiments, when the two electrograms have no other electrogram between them (e.g., in the ordered array). In some embodiments, the term “adjacent”, the term “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the term “adjacent”, the term “proximate”, and the like are not limited to any of the above example definitions, according to some embodiments. In addition, the term “adjacent” and the term “proximate” do not have the same definition, according to some embodiments.

FIG. 1 schematically illustrates a portion of a transducer-activation system or controller system thereof 100 that may be employed to at least select, control, activate, or monitor a function or activation of a number of electrodes or transducers (e.g., ablation transducers configured to cause thermal ablation or ablation transducers configured to cause PFA), according to some embodiments. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components such as data processing device system 110, input-output device system 120, and processor-accessible memory device system 130 form at least part of a controller system (e.g., controller system 324 shown in FIG. 3).

The data processing device system 110 includes one or more data processing devices that implement or execute, in conjunction with other devices, such as those in the system 100, various methods and functions described herein, including those described with respect to methods exemplified in FIGS. 5A-5B. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, quantum, or biological components, or otherwise.

The memory device system 130 includes one or more processor-accessible memory devices configured to store one or more programs and information, including the program(s) and information needed to execute the methods or functions described herein, including those described with respect to FIGS. 5A-5B. The memory device system 130 may be a distributed processor-accessible memory device system including multiple processor-accessible memory devices communicatively connected to the data processing device system 110 via a plurality of computers and/or devices. However, the memory device system 130 need not be a distributed processor-accessible memory system and, consequently, may include one or more processor-accessible memory devices located within a single data processing device or housing. Each of the phrases “processor-accessible memory” and “processor-accessible memory device” and the like is intended to include any processor-accessible data storage device or medium, whether volatile or nonvolatile, electronic, magnetic, optical, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” may include or may be a non-transitory processor-accessible (or computer-readable) data storage medium. In some embodiments, the processor-accessible memory device system 130 may include or may be a non-transitory processor-accessible (or computer-readable) data storage medium system. In some embodiments, the memory device system 130 may include or may be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 may be implemented by a single application-specific integrated circuit (ASIC) in some embodiments.

The input-output device system 120 may include a mouse, a keyboard, a touch screen, another computer, a processor-accessible memory device system, a network-interface card or network-interface circuitry, or any device or combination of devices from which a desired selection, desired information, instructions, or any other data is input to the data processing device system 110. The input-output device system 120 may include a user-activatable control system that is responsive to a user action. The user-activatable control system may include at least one control element that may be activated or deactivated on the basis of a particular user action. The input-output device system 120 may include any suitable interface for receiving information, instructions or any data from other devices and systems described in various ones of the embodiments. In this regard, the input-output device system 120 may include various ones of other systems described in various embodiments. For example, the input-output device system 120 may include at least a portion of a transducer-based device. The phrase “transducer-based device” or “transducer-based device system” is intended to include one or more physical systems that include various transducers. A tissue ablation (e.g., PFA or thermal) device system that includes one or more transducers may be considered a transducer-based device or device system, according to some embodiments.

The input-output device system 120 also may include an image generating device system, a display device system, a speaker or audio output device system, a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In some embodiments, the input-output device system 120 may include a transducer-based device, as discussed above, and in some embodiments, the transducer-based device may act as a device or device system that provides information to, receives instructions or energy from, or both provides information to and receives instructions or energy from the data processing device system 110. In this regard, the input-output device system 120 may include various devices or systems described in various embodiments.

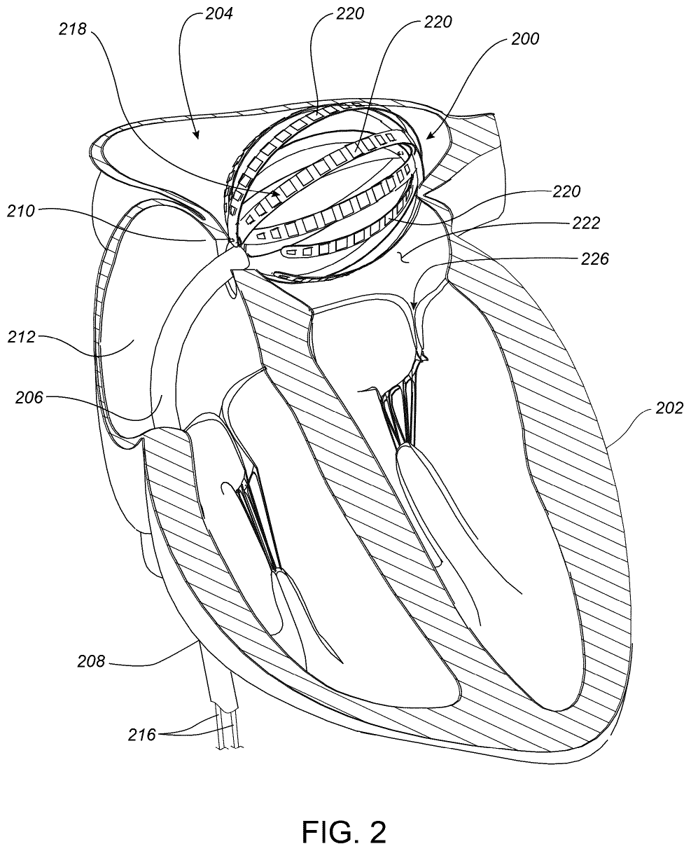

Various embodiments of transducer-based devices are described herein in this disclosure. Some of the described devices are tissue ablation (e.g., PFA or thermal) devices that are percutaneously or intravascularly deployed. Some of the described devices are movable between a delivery or unexpanded configuration (e.g., FIG. 3A discussed below) in which a portion of the device is sized for passage through a bodily opening leading to a bodily cavity, and an expanded or deployed configuration (e.g., FIGS. 2 and 3B discussed below) in which the portion of the device has a size too large for passage through the bodily opening leading to the bodily cavity. An example of an expanded or deployed configuration, in some embodiments, is when the portion of the transducer-based device is in its intended-deployed-operational state, which may be inside the bodily cavity when, e.g., performing an intended therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device is being changed from the delivery configuration to the intended-deployed-operational state to a point where the portion of the device now has a size too large for passage through the bodily opening leading to the bodily cavity.

In some example embodiments, the device includes transducers that sense characteristics (e.g., convective cooling, permittivity, force) that distinguish between fluid, such as a fluidic tissue (e.g., blood), and tissue forming an interior surface of the bodily cavity. Such sensed characteristics can allow a medical system to map the cavity, for example, using positions of openings or ports into and out of the cavity to determine a position or orientation (e.g., pose), or both, of the portion of the device in the bodily cavity. In some example embodiments, the described systems employ a navigation system or electro-anatomical mapping system including electromagnetic-based systems and electropotential-based systems to determine a positioning of a portion of a device in a bodily cavity. In some example embodiments, the described devices are part of a transducer-activation system capable of ablating tissue in a desired pattern within the bodily cavity using various techniques (e.g., via thermal ablation, PFA, etc., according to various embodiments).

In some example embodiments, the devices are capable of sensing various cardiac functions (e.g., electrophysiological activity including intracardiac voltages which form the basis of recorded electrograms according to some embodiments). In some example embodiments, the devices are capable of providing stimulation (e.g., electrical stimulation) to tissue within the bodily cavity. Electrical stimulation may include pacing.

FIG. 2 is a representation of a transducer-based device 200 (which may also be referred to as a medical device) useful in investigating or treating a bodily organ, for example, a heart 202, according to at least one example embodiment.

Transducer-based device 200 can be percutaneously or intravascularly inserted into a portion of the heart 202, such as an intra-cardiac cavity like left atrium 204. In this example, the transducer-based device 200 is part of a catheter 206 inserted via the inferior vena cava 208 and penetrating through a bodily opening in transatrial septum 210 from right atrium 212. (In this regard, transducer-based devices or device systems described herein that include a catheter may also be referred to as catheter devices or catheter-based devices, in some embodiments). In other embodiments, other paths may be taken.

Catheter 206 includes an elongated flexible rod or shaft member appropriately sized to be delivered percutaneously or intravascularly. Various portions of catheter 206 may be steerable. Catheter 206 may include one or more lumens. The lumen(s) may carry one or more communications or power paths, or both. For example, the lumens(s) may carry one or more electrical conductors 216 (two shown). Electrical conductors 216 provide electrical connections to transducer-based device 200 that are accessible externally from a patient in which the transducer-based device 200 is inserted.

Transducer-based device 200 includes a frame or structure 218 which assumes an unexpanded configuration for delivery to left atrium 204. Structure 218 is expanded (e.g., shown in a deployed or expanded configuration in FIG. 2) upon delivery to left atrium 204 to position a plurality of transducers 220 (three called out in FIG. 2) proximate the interior surface formed by tissue 222 of left atrium 204. In some embodiments, at least some of the transducers 220 are used to sense a physical characteristic of a fluid (e.g., blood) or tissue 222, or both, that may be used to determine a position or orientation (e.g., pose), or both, of a portion of a device 200 within, or with respect to left atrium 204. For example, transducers 220 may be used to determine a location of pulmonary vein ostia or a mitral valve 226, or both. In some embodiments, at least some of the transducers 220 may be used to selectively ablate portions of the tissue 222. For example, some of the transducers 220 may be used to ablate a pattern around the bodily openings, ports or pulmonary vein ostia, for instance to reduce or eliminate the occurrence of atrial fibrillation. In some embodiments, at least some of the transducers 220 are used to ablate cardiac tissue. In some embodiments, at least some of the transducers 220 are used to sense or sample intra-cardiac voltage data or sense or sample intra-cardiac electrogram data. In some embodiments, at least some of the transducers 220 are used to sense or sample intra-cardiac voltage data or sense or sample intra-cardiac electrogram data while at least some of the transducers 220 are concurrently ablating cardiac tissue. In some embodiments, at least one of the sensing or sampling transducers 220 is provided by at least one of the ablating transducers 220. In some embodiments, at least a first one of the transducers 220 senses or samples intra-cardiac voltage data or intra-cardiac electrogram data at a location at least proximate to a tissue location ablated by at least a second one of the transducers 220. In some embodiments, the first one of the transducers 220 is other than the second one of the transducers 220.

FIGS. 3A and 3B include a catheter device system (e.g., a portion thereof shown schematically) that includes a medical device 300, according to some embodiments. Medical device 300 may also be referred to as a transducer-based device. All or part of such catheter device system may be all, or part of, a tissue ablation system or sensing system, according to various embodiments. All or part of such catheter device system may be all or part of a medical system, such as system 100 shown in FIG. 1, according to various embodiments. As noted above, the controller 324 may be a particular implementation of the data processing device system 110 shown in FIG. 1.

The medical device 300 (or transducer-based device) may be the same as the transducer-based device 200, although different sizes, numbers of transducers, or types of medical devices, such as balloon catheters, may be implemented. In this regard, medical device 300 includes a plurality of elongate members 304 (not all of the elongate members are called out in FIGS. 3A and 3B) and a plurality of transducers 306 (not all of the transducers are called out in FIGS. 3A and 3B; some of the transducers 306 are called out in FIG. 3B as 306a, 306b, and 306c). FIG. 3A includes a representation of a portion of the medical device 300 in a delivery or unexpanded configuration. FIG. 3B includes a representation of a portion of the medical device 300 in an expanded or deployed configuration. It is noted that, for clarity of illustration, all of the elongate members shown in FIG. 3B are not represented in FIG. 3A. As will become apparent, the plurality of transducers 306 is positionable within a bodily cavity, such as with the medical device 200. For example, in some embodiments, the transducers 306 are able to be positioned in a bodily cavity by movement into, within, or into and within the bodily cavity, with or without a change in a configuration of the plurality of transducers 306. In some embodiments, the transducers of the plurality of transducers 306 are arranged to form a two—or three-dimensional distribution, grid or array of the transducers capable of mapping, ablating, or stimulating an inside surface of a bodily cavity or lumen without requiring mechanical scanning. As shown, for example, in FIG. 3A, the plurality of transducers 306 are arranged in a distribution receivable in a bodily cavity. In FIGS. 3A and 3B, each of at least some of transducers 306 includes a respective electrode 315 (not all of the electrodes 315 are called out in FIGS. 3A and 3B).

The elongate members 304 are arranged in a frame or structure 308 that is selectively moveable between an unexpanded or delivery configuration (e.g., as shown in FIG. 3A) and an expanded or deployed configuration (e.g., as shown in at least FIG. 3B) that may be configured to position elongate members 304 against a tissue surface within the bodily cavity or position the elongate members 304 in the vicinity of the tissue surface. In some embodiments, structure 308 has a size in the unexpanded or delivery configuration suitable for delivery through a bodily opening (e.g., via catheter sheath 312) to the bodily cavity. In various embodiments, catheter sheath 312 typically includes a length sufficient to allow the catheter sheath to extend between a location at least proximate a bodily cavity into which the structure 308 is to be delivered and a location outside a body comprising the bodily cavity. In some embodiments, structure 308 has a size in the expanded or deployed configuration too large for delivery through a bodily opening (e.g., via catheter sheath 312) to the bodily cavity. The elongate members 304 may form part of a flexible circuit structure (e.g., also known as a flexible printed circuit board (PCB) circuit, examples of which are described with respect to FIG. 4, below). The elongate members 304 may include a plurality of different material layers. Each of the elongate members 304 may include a plurality of different material layers. The structure 308 may include a shape memory material, for instance, Nitinol. The structure 308 may include a metallic material, for instance, stainless steel, or non-metallic material, for instance, polyimide, or both a metallic and non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose), or both of structure 308 in the bodily cavity, the requirements for successful ablation of a desired pattern, or the effect that the material may have on electric or magnetic fields to be sensed by the device (e.g., by one or more transducers 306 or one or more magnetic field transducers).

FIG. 4 is a schematic side elevation view of at least a portion of a transducer-based device 400 that includes a flexible circuit structure 401 that is employed to provide a plurality of transducers 406 (two called out) according to an example embodiment. In some embodiments, the flexible circuit structure 401 may form part of a structure (e.g., structure 308) that is selectively movable between a delivery configuration sized for percutaneous delivery and an expanded or deployed configuration sized too large for percutaneous delivery. In some embodiments, the flexible circuit structure 401 may be located on, or form at least part of, a structural component (e.g., elongate member 304) of a transducer-based device system.

The flexible circuit structure 401 can be formed by various techniques including flexible printed circuit techniques. In some embodiments, the flexible circuit structure 401 includes various layers including flexible layers 403a, 403b and 403c (e.g., collectively flexible layers 403). In some embodiments, each of flexible layers 403 includes an electrical insulator material (e.g., polyimide). One or more of the flexible layers 403 can include a different material than another of the flexible layers 403. In some embodiments, the flexible circuit structure 401 includes various electrically conductive layers 404a, 404b, and 404c (collectively electrically conductive layers 404) that are interleaved with the flexible layers 403. In some embodiments, each of the electrically conductive layers 404 is patterned to form various electrically conductive elements. For example, electrically conductive layer 404a is patterned to form a respective electrode 415 of each of the transducers 406. Electrodes 415 have respective electrode edges 415-1 that form a periphery of an electrically conductive surface associated with the respective electrode 415. It is noted that other electrodes employed in other embodiments may have electrode edges arranged to form different electrodes shapes (for example, as shown by electrode edges 315-1 in FIG. 3B).

Electrically conductive layer 404b is patterned, in some embodiments, to form respective temperature sensors 408 for each of the transducers 406 as well as various leads 410a arranged to provide electrical energy to the temperature sensors 408. In some embodiments, each temperature sensor 408 includes a patterned resistive member 409 (two called out) having a predetermined electrical resistance. In some embodiments, each resistive member 409 includes a metal having relatively high electrical conductivity characteristics (e.g., copper). In some embodiments, electrically conductive layer 404c is patterned to provide portions of various leads 410b arranged to provide an electrical communication path to electrodes 415. In some embodiments, leads 410b are arranged to pass though vias in flexible layers 403a and 403b to connect with electrodes 415. Although FIG. 4 shows flexible layer 403c as being a bottom-most layer, some embodiments may include one or more additional layers underneath flexible layer 403c, such as one or more structural layers, such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 4 shows only three flexible layers 403a-403c and only three electrically conductive layers 404a-404c, it should be noted that other numbers of flexible layers, other numbers of electrically conductive layers, or both, can be included.

In some embodiments, electrodes 415 are employed to selectively deliver ablation energy (e.g., thermal ablation energy or PFA energy) to various tissue structures within a bodily cavity (e.g., an intra-cardiac cavity or chamber). The energy delivered to the tissue structures may be sufficient for ablating portions of the tissue structures. The energy delivered to the tissue may be delivered to cause monopolar tissue ablation, bipolar tissue ablation or blended monopolar-bipolar tissue ablation by way of non-limiting example.

Energy that is sufficient for tissue ablation may be dependent upon factors including transducer location, size, shape, relationship with respect to another transducer or a bodily cavity, material or lack thereof between transducers, et cetera. For example, in “RF” ablation, a larger electrode (e.g., an electrode with a relatively large surface area) will achieve a given ablation depth sooner than a smaller electrode. Put differently, a maximum ablation depth of a relatively smaller electrode is typically shallower than that of a relatively larger electrode when ablating under the same control parameters as a relatively larger electrode.

In some embodiments, each electrode 415 is employed to sense or sample an electrical potential in the tissue proximate the electrode 415 at a same or different time than delivering energy sufficient for tissue ablation. In some embodiments, each electrode 415 is employed to sense or sample intra-cardiac voltage data in the tissue proximate the electrode 415. In some embodiments, each electrode 415 is employed to sense or sample data in the tissue proximate the electrode 415 from which an electrogram may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, the resistive members 409 are connected in series to allow electrical current to pass through all of the resistive members 409. In some embodiments, leads 410a are arranged to allow for a sampling of electrical voltage in between resistive members 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by convective cooling effects (e.g., as provided by blood flow).

Referring to FIGS. 3A and 3B, medical device 300 can communicate with, receive power from, or be controlled by a transducer-activation device system 322 according to some embodiments. In some embodiments, at least part of the transducer-activation device system 322 represents one or more particular implementations of the system 100 illustrated in FIG. 1. In some embodiments, elongate members 304 include transducers 306 that are communicatively connected to a data processing device system 310 via electrical connections running within elongate shaft member 314 that are communicatively connected to one or more of electrical leads 317 (e.g., control leads, data leads, power leads or any combination thereof) within elongated cable 316 (only a portion of which is shown in FIGS. 3A and 3B to reveal other structures) terminating at a connector 321 or other interface. The transducer-activation device system 322 may include a controller 324 that includes the data processing device system 310 (e.g., which may be a particular implementation of data processing device system 110 from FIG. 1) and a memory device system 330 (e.g., which may be a particular implementation of the memory device system 130 from FIG. 1) that stores data and instructions that are executable by the data processing device system 310 to process information received from medical device 300 or to control operation of medical device 300, for example, activating various selected transducers 306 to ablate tissue and control a user interface (e.g., of input-output device system 320) according to various embodiments. Controller 324 may include one or more controllers.

Transducer-activation device system 322 includes an input-output device system 320 (e.g., which may be a particular implementation of the input-output device system 120 from FIG. 1) communicatively connected to the data processing device system 310 (e.g., via controller 324 in some embodiments). Input-output device system 320 may include a user-activatable control that is responsive to a user action. Input-output device system 320 may include one or more user interfaces or input/output (I/O) devices, for example, one or more display device systems 332, speaker device systems 334, one or more keyboards, one or more mice (e.g., mouse 335), one or more joysticks, one or more track pads, one or more touch screens or other transducers to transfer information to, from, or both to and from a user, for example a care provider such as a physician or technician. For example, output from a mapping process may be displayed by a display device system 332. Input-output device system 320 may include one or more user interfaces or input/output (I/O) devices, for example, one or more display device systems 332, speaker device systems 334, keyboards, mice, joysticks, track pads, touch screens or other transducers employed by a user to indicate a particular selection or series of selections of various graphical information. Input-output device system 320 may include a sensing device system 325 configured to detect various characteristics including, but not limited to, at least one of tissue characteristics (e.g., electrical characteristics such as tissue impedance, electric potential of a tissue surface, tissue conductivity, tissue type, tissue thickness) and thermal characteristics such as temperature. In this regard, the sensing device system 325 may include one, some, or all, of the transducers 306 (or 220 in FIG. 2 or 406 of FIG. 4) of the medical device 300, including the internal components of such transducers shown in FIG. 4, such as the electrodes 415 and temperature sensors 408.

Transducer-activation device system 322 may also include an energy source device system 340 including one or more energy source devices connected to transducers 306. In this regard, although FIGS. 3A and 3B show a communicative connection between the energy source device system 340 and the controller 324 (and its data processing device system 310), the energy source device system 340 may also be connected to the transducers 306 via a communicative connection that is independent of the communicative connection with the controller 324 (and its data processing device system 310). For example, the energy source device system 340 may receive control signals via the communicative connection with the controller 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through catheter body or elongate shaft member 314, elongated cable 316 or catheter sheath 312) that does not pass through the controller 324. In this regard, the energy source device system 340 may provide results of its delivering energy to, receiving energy from, or both delivering energy to and receiving energy from one or more of the transducers 306 to the controller 324 (and its data processing device system 310) via the communicative connection between the energy source device system 340 and the controller 324.

The energy source device system 340 may, for example, be connected to various selected transducers 306 to selectively provide energy in the form of electrical current or power, light, low temperature fluid, or another form to the various selected transducers 306 to cause ablation of tissue. The energy source device system 340 may, for example, selectively provide energy in the form of electrical current to various selected transducers 306 and measure a temperature characteristic, an electrical characteristic, or both at a respective location at least proximate each of the various transducers 306. The energy source device system 340 may include various electrical current sources or electrical power sources as energy source devices. In some embodiments, an indifferent electrode 326 is provided to receive at least a portion of the energy transmitted by at least some of the transducers 306. Consequently, although not shown in FIGS. 3A and 3B, the indifferent electrode 326 may be communicatively connected to the energy source device system 340 via one or more communication lines in some embodiments. In addition, although shown separately in each of FIGS. 3A and 3B, indifferent electrode 326 may be considered part of the energy source device system 340 in some embodiments. In various embodiments, indifferent electrode 326 is positioned on an external surface (e.g., a skin-based surface) of a body that comprises the bodily cavity into which at least transducers 306 are to be delivered.

It is understood that input-output device system 320 may include other systems. In some embodiments, input-output device system 320 may optionally include energy source device system 340, medical device 300 or both energy source device system 340 and medical device 300 by way of non-limiting example. Input-output device system 320 may include the memory device system 330 in some embodiments.

In other example embodiments, other structures besides those shown in FIGS. 2, 3A, 3B, and 4 may be employed to support or carry transducers of a transducer-based device such as a transducer-based catheter. For example, an elongated catheter member may be used to distribute the transducers in a linear or curvilinear array. Basket catheters or balloon catheters may be used to distribute the transducers in a two-dimensional or three-dimensional array.

FIGS. 5A and 5B include respective data generation and flow diagrams, which may implement various embodiments of methods 500 (with FIG. 5B showing details of one of the blocks (block 506) in FIG. 5A, according to some embodiments) by way of associated computer-executable instructions according to some example embodiments. In various example embodiments, a memory device system (e.g., memory device systems 130, 330) is communicatively connected to a data processing device system (e.g., data processing device systems 110 or 310, otherwise stated herein as “e.g., 110, 310”) and stores a program executable by the data processing device system to cause the data processing device system to execute various embodiments of methods 500 via interaction with at least, for example, a transducer-based device (e.g., transducer-based device 200, 300, or 400, in some embodiments). In these various embodiments, the program may include instructions configured to perform, or cause to be performed, various embodiments of methods 500. In some embodiments, the methods 500 may include a subset of the associated blocks or additional blocks than those shown in FIGS. 5A and 5B. For example, a particular one of methods 500 may adopt the actions of block 502a, a particular one of methods 500 may adopt the actions of block 502b, a particular one of methods 500 may adopt the actions of block 502a and block 502b, and a particular one of methods 500 may adopt other actions described herein other than those of block 502a and block 502b. The broken line arrow between blocks 502a and 502b indicates an optional flow where, in some embodiments, the actions of block 502b are executed or configured to be executed after block 502a, whereas, in other embodiments, the actions of block 502a and 502b may occur in a different sequence, independently, or one without the other. Blocks 504a and 504b may be treated in a similar manner according to various embodiments. In this regard, some embodiments of method 500 may include the sequence of blocks 502a, 504a, and 506 without the sequence of blocks 502b, 504b, and 506, or vice versa. As noted above, in some embodiments, the methods 500 may include a different sequence than those indicated between various ones of the associated blocks shown in FIGS. 5A and 5B. In FIG. 5B, blocks 506a and 506b are illustrated in broken line. Such broken line blocks illustrate possible implementation details for block 506, according to some embodiments. The features recited by any block are not intended to be exclusive, and the adopting of any recited features of any particular block in a particular embodiment does not prevent the inclusion of any other features, according to some embodiments, unless the features cannot work together (i.e., are mutually exclusive).

According to some embodiments, methods 500 may include block 502a associated with computer-executable instructions (e.g., reception instructions provided by a program) configured to cause a data processing device system (e.g., 110, 310) to receive input via an input-output device system (e.g., 120, 320), the input indicating a selection of a first transducer set of a plurality of transducers (e.g., transducers 220, 306, 406) of at least a portion of a transducer-based device (e.g., 200, 300, or 400). According to various embodiments, the at least the portion of the transducer-based device is positionable within a bodily cavity. For example, the at least the portion of the transducer-based device may include structure (e.g., structure 218 or 308) that is configured to support various transducers and to be deliverable to, or positionable within, a bodily cavity. According to various embodiments, each transducer in the first transducer set may be configured to transmit tissue ablative energy. For example, in some embodiments, each transducer in the first transducer set is configured to transmit particular energy configured to cause pulsed field ablation of tissue. In some embodiments, each transducer in the first transducer set is configured to transmit particular energy configured to cause thermal ablation of tissue. In some embodiments, each transducer in the first transducer set may be configured to record a respective electrogram. For example, in some embodiments, each transducer in the first transducer set may include an electrode (e.g., 315, 415), each electrode configured to (a) transmit tissue ablative energy, (b) record electrograms (e.g., as described above in this disclosure with respect to FIG. 4), or (a) and (b), according to various embodiments. According to various embodiments, the transducers of the plurality of transducers (e.g., transducers 220, 306, 406) may be individually selectable. According to various embodiments, the transducers of the plurality of transducers (e.g., transducers 220, 306, 406) may be individually addressable for identification for selection, ablation, or sensing. According to some embodiments, the received input (e.g., received per block 502a) includes user-based input indicating the selection of the first transducer set. The selection of a particular transducer of the plurality of transducers (e.g., transducers 220, 306, 406) for inclusion in the first transducer set may be made on the basis of various criteria, such as a degree of tissue contact exhibited by the transducers, according to various embodiments. In this regard, those transducers exhibiting a sufficient degree of tissue contact (or a sufficient degree of proximity in some embodiments) to allow for successful tissue ablation may be selected for inclusion in the first transducer set, in some embodiments.

For example, according to various embodiments, different parts of the at least the portion of the transducer-based device may be manipulable to, in turn, manipulate various ones of the plurality of transducers (e.g., transducers 220, 306, 406) into various degrees of contact with a tissue wall within a patient's body. According to various embodiments, each transducer of at least some transducers of the plurality of transducers (e.g., transducers 220, 306, 406) may be configured at least to sense a degree of contact between the transducer and the tissue wall. In some embodiments, each particular transducer of at least some transducers of the plurality of transducers may be configured to sense or detect a degree or amount of transducer-to-tissue contact between at least a portion of the particular transducer and the tissue wall. Various methods may be executed to determine the degree or amount of transducer-to-tissue contact including, by way of non-limiting example, techniques including sensing impedance, sensing permittivity, sensing the presence or absence of flow of a fluid (e.g., a bodily fluid), or by sensing contact force or pressure. U.S. Pat. No. 8,906,011, issued Dec. 9, 2014 (Gelbart et al.), describes example transducer sensing techniques to sense tissue contact. In some embodiments, the tissue-contacting portion of the transducer itself directly senses the degree of tissue contact. In some embodiments, a portion of the transducer other than the tissue-contacting portion of the transducer is configured to sense the degree of contact between the tissue wall and the tissue-contacting portion of the transducer. In some embodiments, the tissue-contacting portion of the transducer is provided by an electrode.

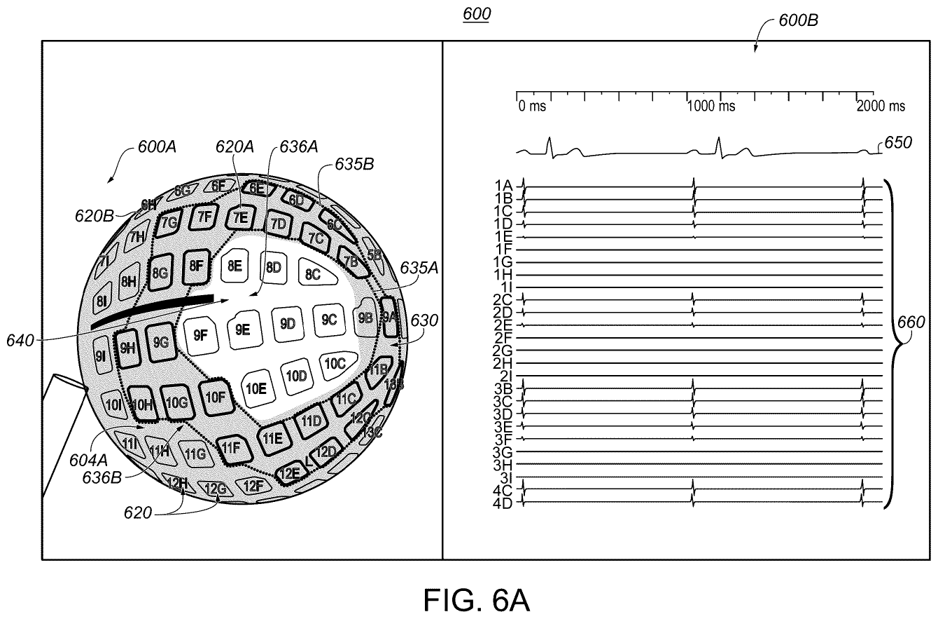

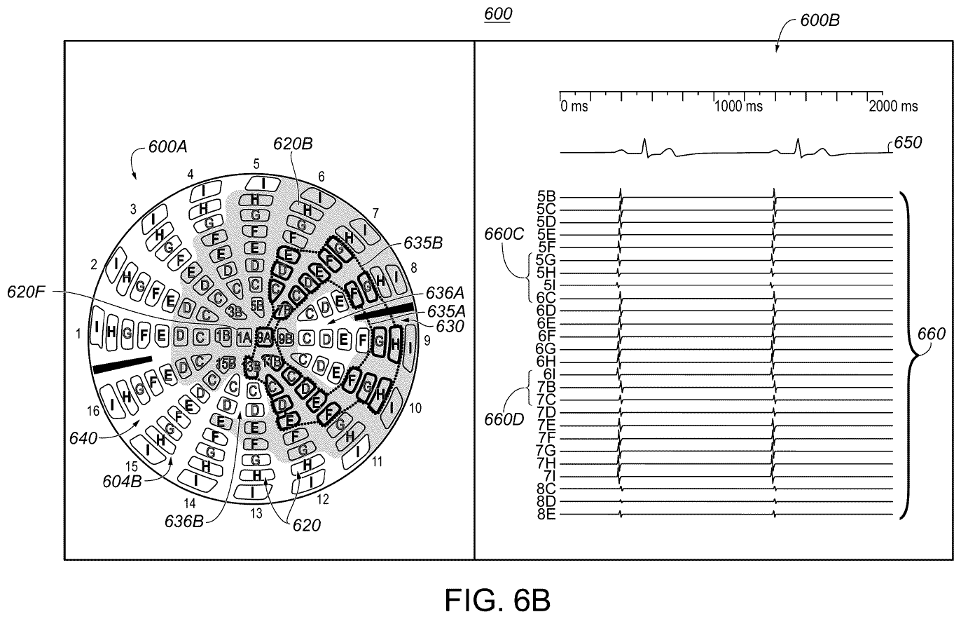

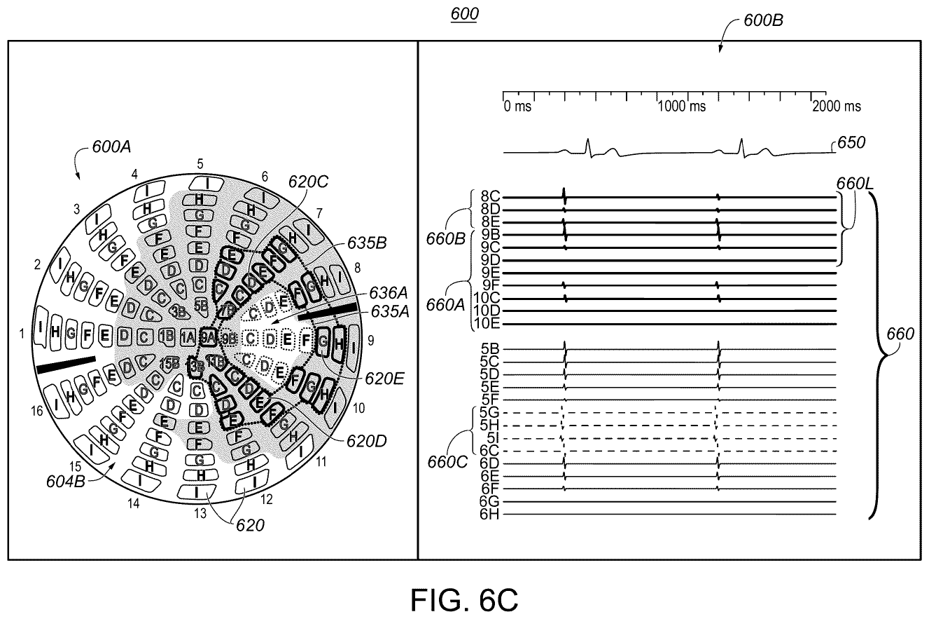

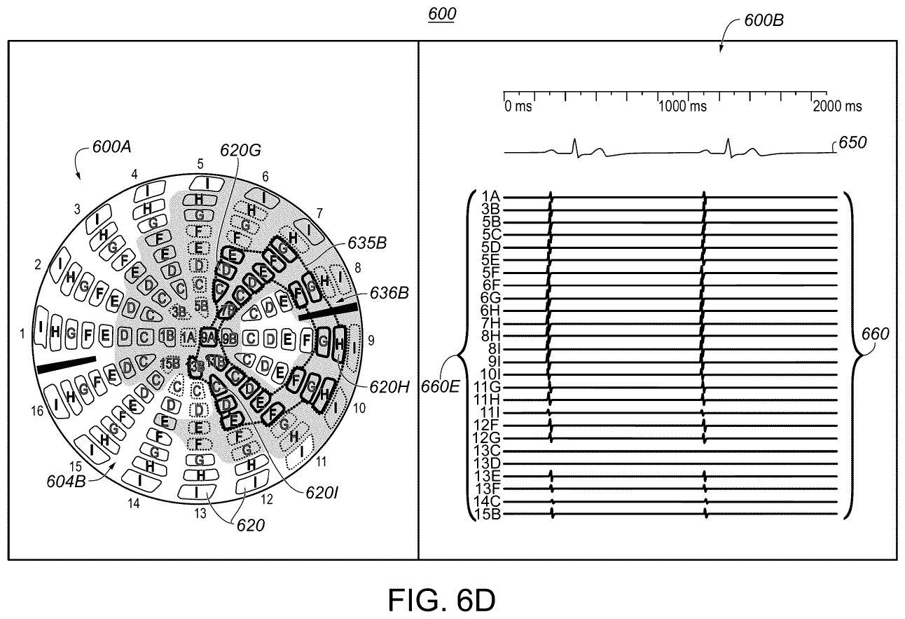

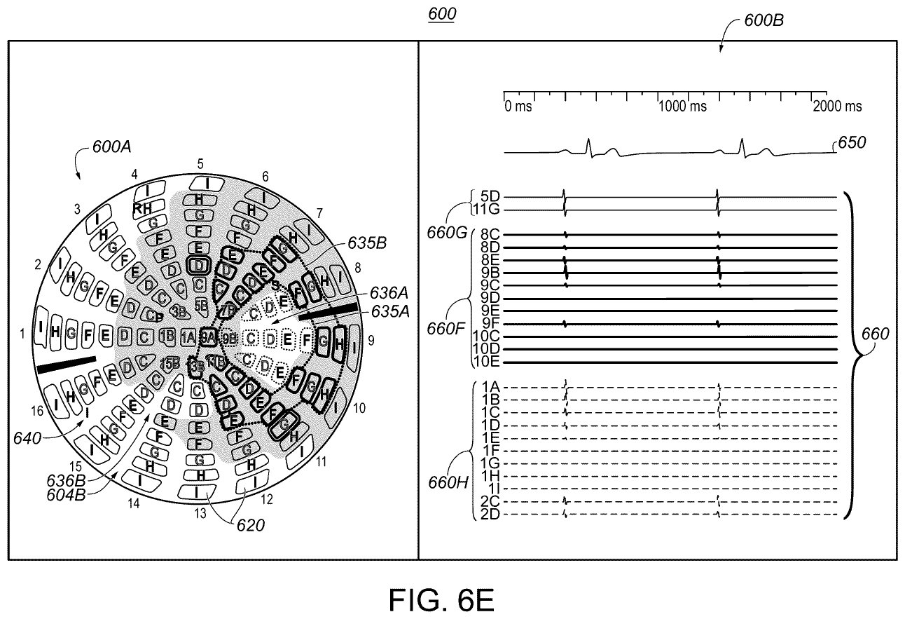

According to some embodiments, at least some transducers of the plurality of transducers of the transducer-based device (e.g., 200, 300, 400) may be configured to provide a plurality of contact signal sets to the controller 324 or its data processing device system 310. The plurality of contact signal sets may be referred to as degree of transducer-to-tissue contact information in some embodiments. In this regard, each contact signal set may provide or indicate a degree of transducer-to-tissue contact between each transducer (e.g., a transducer 220, 306, 406) and a tissue surface in the bodily cavity. According to various embodiments, the degree of transducer-to-tissue contact information may be communicated to a user (e.g., a health care practitioner) to assist the user in selecting the first transducer set from the plurality of transducers (e.g., transducers 220, 306, 406). In this regard, the input-output device system 120, 320 may include any suitable interface for outputting information (e.g., transducer-to-tissue contact information), instructions, or data to other devices and systems described in various ones of the embodiments. In this regard, the input-output device system 120, 320 may include various other devices or systems described in various embodiments. In some embodiments, the input-output device system 120, 320 may include one or more display devices that display one or more of the graphical interfaces and graphical representations of FIGS. 6A-6E (collectively, FIG. 6).

According to various embodiments, the data-processing device system 110, 310 may make a machine-based selection of the first transducer set from the plurality of transducers (e.g., transducers 220, 306, 406) based at least on the degree of transducer-to-tissue contact information.

FIGS. 6A-6E show, according to some embodiments, an example of a particular graphical representation 600 (or graphical interface since it may be configured to receive user input in some embodiments) that may be displayed, according to some embodiments, as per block 506 of methods 500. According to some embodiments, each of FIGS. 6A-6E includes a first portion 600A and a second portion 600B of the graphical representation 600. The first portion 600A of the graphical representation 600 depicts information related to selection of the first transducer set. The second portion 600B of the displayed graphical representation 600 displays, among other things, a plurality of electrograms which are discussed in more detail below. It is noted that, although the first portion 600A and the second portion 600B are shown as forming the entirety of displayed graphical representation 600 in FIGS. 6A-6E, such need not be the case in other embodiments where the first and second portions 600A, 600B form parts of different graphical representations or include additional features. The graphical representation 600, or different portions thereof, may be displayed via multiple display devices in some embodiments.

The first portion 600A of the displayed graphical representation 600 may be used to visually indicate the selected first transducer set, according to various embodiments. For example, in some embodiments, a list of graphical elements identifying particular ones of the plurality of transducers (e.g., transducers 220, 306, 406) as selected for inclusion in the first transducer set may be displayed. In some embodiments, the first transducer set may be arranged according to a first spatial distribution, and first portion 600A of the graphical representation 600 may include a set of transducer graphical elements (e.g., bolded transducer graphical elements 620 in at least FIG. 6A, such as bold transducer graphical element 620A), where each transducer graphical element graphically represents a respective transducer in the selected first transducer set, and the transducer graphical elements in the set of transducer graphical elements are graphically arranged according to a second spatial distribution that is consistent with the first spatial distribution. In some embodiments, the entirety of the plurality of transducers is arranged according to a first spatial distribution, and each transducer is graphically represented by a transducer graphical element 620, the transducer graphical elements graphically arranged according to a second spatial distribution that is consistent with the first spatial distribution.

(Only two transducer graphical elements 620 and transducer graphical element 620A are called out in FIG. 6A for clarity.) In some embodiments, the arrangement of the transducer graphical elements is graphically represented in a three-dimensional manner (for example, as shown in FIG. 6A). In FIG. 6A, a three-dimensional graphical representation 604A including a three-dimensionally represented arrangement of transducer graphical elements 620 corresponds to at least a portion of the transducer-based device that is similar to the spherical or quasi-spherical arrays of transducers shown in FIGS. 2 and 3B.

In some embodiments, the arrangement of the transducer graphical elements 620 is graphically represented in a two-dimensional manner. For example, in FIGS. 6B-6E, the first portion 600A of the graphical representation 600 includes a two-dimensional graphical representation 604B of the at least the portion of the transducer-based device, depicted in a two-dimensional graphical manner, according to some embodiments. In this regard, the at least the portion of the transducer-based device is similar to the spherical or quasi-spherical arrays of transducers shown in FIGS. 2 and 3B. In this regard, the two-dimensional graphical representation 604B may be considered to correspond to or represent the at least the portion of the medical device 200, 300, 400 or a medical device similar thereto, according to various embodiments. In some embodiments, the transducer graphical elements 620 in FIG. 6 may correspond to transducers 220, 306, 406.

In various embodiments, the two-dimensional graphical representation (e.g., two-dimensional graphical representation 604B shown in FIGS. 6B-6E) of the at least the portion of the medical device (e.g., 200, 300, 400) corresponds to or represents a three-dimensional shape or form of the at least the portion of the medical device (e.g., 200, 300, 400). Various two-dimensional graphical representations are possible in various embodiments. For instance, in some embodiments, the two-dimensional graphical representation 604B maps three-dimensional surface portions of the at least the portion of the medical device onto a two-dimensional coordinate frame. For example, in some embodiments associated with FIGS. 6, the surface portions of the at least the portion of the medical device 200, 300, 400 may include surface portions (e.g., electrodes 315, 415) of various transducers 220, 306, 406 provided by the at least the portion of the medical device. In FIGS. 6B-6E, transducer graphical elements 620 representative of the transducers are arranged graphically in a two-dimensional distribution in the first portion 600A of the particular graphical representation 600. In this regard, in some embodiments in which the at least the portion of the medical device 200, 300, 400 includes a plurality of transducers, the two-dimensional graphical representation 604B may map information indicating a three-dimensional spatial distribution of the plurality of transducers projected onto a two-dimensional coordinate frame. In some embodiments, a plurality of transducer graphical elements (e.g., 620) may be arranged in the particular graphical representation 600 in a particular spatial distribution representing the three-dimensional distribution of transducers (e.g., 220, 306, 406) of the at least the portion of the medical device distorted onto a two-dimensional plane to form the two-dimensional graphical representation. In this regard, in some embodiments, the two-dimensional graphical representation 604B of the three-dimensional distribution of transducers (e.g., 220 or 306) distorted onto a two-dimensional plane is not merely an isometric or other perspective view of the three-dimensional distribution of transducers, because such an isometric or other perspective view may be considered a three-dimensional graphical representation. Accordingly, in some embodiments, the two-dimensional graphical representation 604B may represent a map including spatial distortion caused by mapping, e.g., a curved three-dimensional surface distorted onto a flat two-dimensional surface. In the example of at least FIG. 6B, such distortion is viewable by the distorted transducer (e.g., electrode) sizes of some of the transducer graphical elements 620 along the outer edge of the two-dimensional graphical representation 604B.

The two-dimensional graphical representation 604B may be generated according to a conformal map or projection, such as a Mercator map or projection, a transverse Mercator map or projection (also known as a Cassini projection), or other three-dimensional-to-two-dimensional mapping or projection, known in the art, according to some embodiments. Sec, e.g., U.S. Pat. No. 10,368,936, issued Aug. 6, 2019 (Brewster et al.) in relation to various mapping techniques. According to various embodiments, a conformal mapping is a function that preserves local angles. For example, according to some embodiments, when a particular spatial relationship between the plurality of transducers 220, 306, 406 is conformally mapped to the particular graphical representation 600, an angle defined between a group of transducers (e.g., 220, 306) according to the particular spatial relationship is preserved between the corresponding group of transducer graphical elements 620. In FIGS. 6B-6E, the transducer graphical elements 620 are mapped in a two-dimensional projection that approximates a Lambert azimuthal projection. In this two-dimensional projection, the transducer graphical elements 620 representing all of the transducers 306, 406 radiate at least in part along a radial line from a center of the projection.

According to various embodiments associated with FIGS. 6, transducers 220, 306 are distributed over each of two hemispherical regions provided by the at least the portion of the medical device, and the transducer graphical elements 620 representing all of the transducers 220, 306 are graphically depicted in a two-dimensional distribution in which all the transducer graphical elements 620 are radially distributed along the various radial lines extending from a particular region in the two-dimensional distribution that corresponds to a pole of one of the two hemispherical regions. Each transducer graphical element 620 is identified by a letter identifier and a numerical identifier, where the numerical identifier indicates along which radial line the transducer graphical element is located on. For instance, transducer graphical element 620B in FIG. 6B is identified by letter “H” and number “6”, where the number “6” indicates that the transducer graphical element 620B is located on radial line “6”. According to various embodiments, the same “letter” and “numerical” identifiers are employed to identify the transducer graphical elements 620 in the three-dimensional graphical representation 604A shown in the first portion 600A of FIG. 6A.

According to some embodiments, each radial line may correspond to a “line of longitude” associated with the at least the portion of the transducer-based device, each line of longitude extending between two poles of the at least the portion of the transducer-based device, and a subset of the plurality of transducers (e.g., 220, 306, 406) distributed along each line of longitude. In some embodiments, and with reference to FIG. 3B, each radial line corresponds to at least a portion of a corresponding elongate member 304. In some embodiments, the two-dimensional graphical representation need not be a projection or mapping from a three-dimensional model, and may merely be any two-dimensional graphical representation, e.g., including an arrangement of transducers.

According to some embodiments, various features that are mapped onto the two-dimensional graphical representation (e.g., 604B) may have a distorted appearance (for example, at least some of the transducer graphical elements 620 may have a distorted appearance). In some embodiments, a two-dimensional graphical representation (e.g., 604B) may map three-dimensional tissue surface portions within the bodily cavity onto a two-dimensional coordinate frame in a manner similar to that described by Raymond E. Ideker, M. D., Ph.D., et al. in the document “A Computerized Method for the Rapid Display of Ventricular Activation During the Intraoperative Study of Arrhythmias”, in the journal Circulation, vol. 59, No. 3, pages 449-458 (Mar. 1, 1979). In this document, Ideker et al. disclose various two-dimensional surface maps in which a total heart surface is depicted two-dimensionally as if the ventricles were folded out after an imaginary cut was made from the crux to the apex.

In some particular embodiments in which the at least the portion of the medical device (e.g., medical device 200, 300, 400) includes a plurality of transducers (e.g., transducers 220, 306, 406), the two-dimensional graphical representation (e.g., two-dimensional graphical representation 604B) maps information indicating a three-dimensional spatial distribution of transducer-to-tissue contact information onto a two-dimensional coordinate frame. For example, in FIGS. 6A and 6B, regions 630 (i.e., represented by shaded portions of the three-dimensional graphical representation 604A and two-dimensional graphical representation 604B, respectively) indicate greater degrees of transducer-to-tissue contact than the unshaded regions 640, which indicate little or no transducer-to-tissue contact. Although FIGS. 6A and 6B illustrate shaded regions 630 (and although FIGS. 6C-6E also show such shaded regions without a reference numeral) with a solid uniform gray color for ease of illustration to indicate in an uncomplicated visual manner a relatively greater degree of tissue contact than unshaded regions 640, it should be noted that individual transducers may be configured to sense various degrees of tissue contact (instead of a binary value of sufficient contact or insufficient contact) and, therefore, visual indications of transducer contact may be represented using a gradient of colors or shades, for instance, to represent various degrees of tissue contact, according to some embodiments.

Referring to FIGS. 6, the selection of the first transducer set as per block 502a in FIG. 5A is indicated by particular ones of the transducer graphical elements 620 whose outlines have been bolded (like transducer graphical element 620A in at least FIG. 6A, and as compared to other transducer graphical elements 620 which correspond to transducers that have not been selected for inclusion in the first transducer set and do not have bolded outlines) according to various embodiments. It is noted that alternative or additional visual characteristics may be imparted on the transducer graphical elements 620 to indicate various statuses (e.g., a selected status) according to various embodiments. In FIGS. 6, additional or alternate markers 635A and 635B may be employed, according to some embodiments. According to some embodiments, marker 635A indicates a boundary of region 636A of the graphical representations 604A, 604B that is interior at least some of the transducer graphical elements 620 corresponding to the first transducer set. According to some embodiments, marker 635B indicates a boundary of a region 636B of the graphical representations 604A, 604B that is exterior at least some of the transducer graphical elements 620 corresponding to the first transducer set. In this regard, the markers 635A and 635B collectively form the inner and outer boundaries of the region encompassing the bold-outlined transducer graphical elements 620 corresponding to the first transducer set, according to some embodiments.

According to various embodiments, the selected transducers correspond to particular ones of the transducers that exhibit higher degrees of transducer-to-tissue contact (e.g., as represented by the corresponding transducer graphical elements 620 being located in a shaded region 630 in the graphical representation (e.g., 604A, 604B). In various embodiments associated with FIGS. 6, transducers associated with transducer graphical elements identified as “6C”, “6D”, “6E”, “7B”, “7C”, “7D”, “7E”, “7F”, “7G”, “8F”, “8G”, “9A”, “9G”, “9H”, “10F”, “10G”, “10H”, “11B”, “11C”, “11D”, “11E”, “11F”, “12C”, “12D”, “12E”, and “13B” are selected for inclusion in the first transducer set, as indicated by the bold outlines of the respective transducer graphical elements 620.

In some embodiments, the selection of the first transducer set may be based on particular user-input that selects particular transducers of the plurality of transducers by way of, via the input-output device system 120, 320, a selection of respective ones of the transducer graphical elements 620. For example, in some embodiments, a transducer graphical element 620 may be selected via the use of a keyboard, mouse cursor, or touch screen by way of non-limiting example. The selection of particular transducers for inclusion in the first transducer set may be based on various factors. For example, in some embodiments, the selection of particular transducers for inclusion in the first transducer set may be made at least in part on the basis of transducer data (e.g., the transducer-to-tissue contact information shown in FIG. 6 via the shaded and non-shaded regions 630, 640 respectively). In some embodiments, the transducer data may be provided by at least some of the plurality of transducers (e.g., transducers 220, 306, 406).

In some embodiments, the input received via block 502a in FIG. 5A includes machine-based input indicating the selection of the first transducer set. Machine-based or automatic selection of particular transducers of the plurality of transducers (e.g., transducers 220, 306, 406) may be accomplished in various manners, e.g., based on an analysis of information, such as sensed degree of tissue-contact information, sensed temperature information, location information from a navigation system. For instance, the data processing device system 110, 310 may be configured to analyze at least some of such information to automatically select transducers (e.g., transducers 220, 306, 406) that surround and are adjacent to an anatomical feature, such as a port or opening in the bodily cavity. In this regard, for example, U.S. Pat. No. 11,633,238, issued Apr. 25, 2023, which is hereby incorporated herein by reference in its entirety, describes program instructions configured to cause a data processing device system to determine an ablation path based at least on a determination of a proximity of various ones of non-anatomical feature-specific features to various ones of anatomical feature-specific transducers associated with an anatomical feature corresponding to a selected anatomical feature region.

Referring to FIG. 5A, methods 500 may include block 504a associated with computer-executable instructions (e.g., reception instructions provided by a program) configured to cause the data processing device system (e.g., 110, 310) to determine, at least in response to the selection of the first transducer set (e.g., per block 502a), a second transducer set of the plurality of transducers (e.g., transducers 220, 306, 406) satisfying a determined positional relationship with respect to the first transducer set. According to various embodiments, each transducer in the second transducer set may be configured to record a respective electrogram. According to some embodiments, each transducer in the second transducer set may be configured to transmit tissue ablative energy. In some embodiments, each transducer in the first transducer set and each transducer in the second transducer set may include an electrode. According to various embodiments, the second transducer set is mutually exclusive with the first transducer set. In such embodiments, any transducer of the plurality of transducers (e.g., transducers 220, 306, 406) forming part of the first transducer set does not form part of the second transducer set, and vice versa. According to various embodiments, the determination of the second transducer set is a machine-based or an automatic determination made by the data processing device system 110, 310. In this regard, as described in more detail herein, the data processing device system (e.g., 110, 310) may be configured to determine the second transducer set as satisfying a predetermined positional relationship with respect to the first transducer set.

Referring to FIGS. 6, the selected transducer graphical elements 620 (i.e., indicated by bolded outlines, according to some embodiments) indicate that the first transducer set includes at least three transducers of the plurality of transducers (e.g., at least the transducers 220, 306, 406 corresponding to transducer graphical elements 620C, 620D, and 620E in FIG. 6C), according to some embodiments.

According to some embodiments, the at least three transducers may be arranged at non-colincar locations bounding (e.g., forming at least part of a boundary of) a region of space (graphically represented by region 636A) interior (e.g., at least in part on a concave side) of the non-colincar locations of the at least three transducers. For example, at least in FIG. 6C, the transducer graphical elements 620C, 620D, and 620E identified as “7D”, “11E”, and “9G”, respectively, correspond to three of the plurality of transducers (e.g., transducers 220, 306, 406) that are located at non-colincar locations that bound the interior region of space graphically represented by, or corresponding to, graphical region 636A. According to various embodiments, the data processing device system 110, 310 may be configured by the program at least to determine, for inclusion in the second transducer set, particular transducers of the plurality of transducers (e.g., transducers 220, 306, 406) located at least in part or entirely in the region of space (e.g., graphically represented by graphical region 636A) as satisfying the determined positional relationship with respect to the first transducer set, at least in some embodiments in which the determined positional relationship is, e.g., within a region (e.g., represented by graphical region 636A) interior (e.g., at least in part on a concave side) of the first transducer set. For example, in FIG. 6C, transducer graphical elements 620 identified as “8C”, “8D”, “8E”, “9B”, “9C”, “9D”, “9E”, “9F”, “10C”, “10D”, and “10E” correspond to the transducers of the plurality of transducers (e.g., transducers 220, 306, 406) determined to form the second transducer set according to various embodiments in which the determined positional relationship is, e.g., within a region (e.g., region 636A) interior (e.g., at least in part on a concave side) of the first transducer set. In FIG. 6C, the transducers selected for inclusion in the second transducer set have their corresponding transducer graphical elements 620 shown with a dotted outline.

According to some embodiments, the at least three transducers may be arranged at non-colincar locations bounding (e.g., forming at least part of a boundary of) a region of space (graphically represented by region 636B) exterior (e.g., at least in part on a convex side) of the non-colinear locations of the at least three transducers. For example, at least in FIG. 6D, the transducer graphical elements 620G, 620H, and 620I identified as “6D”, “9H” and “12C”, respectively, correspond to three of the plurality of transducers (e.g., transducers 220, 306, 406) that are located at non-colinear locations that bound the exterior region of space corresponding to graphical region 636B. According to various embodiments, the data processing device system 110, 310 may be configured by the program at least to determine, for inclusion in the second transducer set, particular transducers of the plurality of transducers (e.g., transducers 220, 306, 406) located at least in part or entirely in the region of space (e.g., represented by graphical region 636B) as satisfying the determined positional relationship with respect to the first transducer set, at least in some embodiments in which the determined positional relationship is, e.g., within a region (e.g., represented by graphical region 636B) exterior (e.g., at least in part on a convex side) of the first transducer set. For example, in FIG. 6D, transducer graphical elements 620 identified as “1A”, “3B”, “5B”, “5C”, “5D”, “5E”, “5F”, “6F”, “6G”, “6H”, “7H”, “8H”, “81”, “9I”, “101”, “11G”, “11H”, “111”, “12F”, “12G”, “13C”, “13D”, “13E”, “13F”, “14C”, and “15B” correspond to the transducers of the plurality of transducers (e.g., transducers 220, 306, 406) determined to form the second transducer set according to various embodiments in which the determined positional relationship is, e.g., within a region (e.g., region 636B) exterior (e.g., at least in part on a convex side) of the first transducer set. In some embodiments, the determined positional relationship may require adjacency or a certain proximity or distance (e.g., within two or some other particular number of transducers) to a transducer in the first transducer set. To elaborate, in FIG. 6D, not all transducers exterior of the first transducer set are selected for inclusion in the second transducer set, but only those transducers that are exterior of the first transducer set and are adjacent a transducer in the first transducer set are selected for including in the second transducer set, according to some embodiments.

At least in FIGS. 6C and 6D, the transducer graphical elements 620 corresponding to the second transducer set are indicated with a dotted outline according to various embodiments, although other additional or alternate visual characteristic sets may be employed in other embodiments. In some embodiments, the visual characteristics of the transducer graphical elements 620 corresponding to the transducers of the first transducer set are different than the visual characteristics of the transducer graphical elements 620 corresponding to the transducers of the second transducer set. In some embodiments, the visual characteristics of the transducer graphical elements 620 corresponding to the transducers of the second transducer set undergo no changes from a first state just prior to the determination of the second transducer set to a second state just after the determination of the second transducer set.

In some embodiments, every transducer of the plurality of transducers (e.g., transducers 220, 306, 406) that is determined as satisfying the determined positional relationship with respect to the first transducer set is determined for inclusion in the second transducer set (for example, as shown in FIG. 6C). In some embodiments, each of at least some, but not all, of the transducers of the plurality of transducers (e.g., transducers 220, 306, 406) that are determined as satisfying the determined positional relationship with respect to the first transducer set are determined for inclusion in the second transducer set (for example, as shown in FIG. 6D). According to various embodiments, a location of each transducer in the first transducer set is arranged at a respective location, the respective locations of at least some of the transducers in the first transducer set bounding (e.g., forming at least part of a boundary of) the region of space. For example, at least in FIG. 6C, at least some of the transducer graphical elements 620 associated with the first transducer set bound (e.g., form a boundary of) the graphical region 636A corresponding to the interior region of space while in FIG. 6D, at least some of the transducer graphical elements 620 associated with the first transducer set bound (e.g., form a boundary of) the graphical region 636B corresponding to the exterior region of space.

According to some embodiments associated with various ones of FIGS. 6, the transducer graphical elements 620 associated with the at least some of the transducers in the first transducer set may be located at least proximate or abutting graphical marker 635A or 635B. In some embodiments, various transducers in the first transducer set are distributed to at least partially surround a particular anatomical feature in the bodily cavity. For example, in some embodiments, the bodily cavity is a cardiac cavity, and the particular anatomical feature is a bodily opening in the cardiac cavity. In various ones of FIGS. 6, the transducer graphical elements 620 corresponding to the first transducer set surround a particular graphical region that has little shading indicating that the graphical region corresponds to a region of low transducer-to-tissue contact, which may be provided in some embodiments in which the at least the portion of the transducer-based device is abutted against a tissue surface surrounding a bodily opening. In this regard, the graphical region 640 corresponding to an unshaded region indicating little or no transducer-to-tissue contact in that region, may correspond to such a bodily opening of the bodily cavity.

Referring to block 504a in FIG. 5A, the positional relationship with respect to the first transducer set may be determined prior to the selection of the first transducer set. In some embodiments, the positional relationship is defined and programmed into the data processing device system 110, 310 prior to the selection of the first transducer set. In this regard, in some embodiments, the positional relationship with respect to the first transducer set is determined based at least in part on particular data stored in the memory device system 130, 330, the particular data indicating a predetermined positional relationship with respect to at least a selected transducer set.

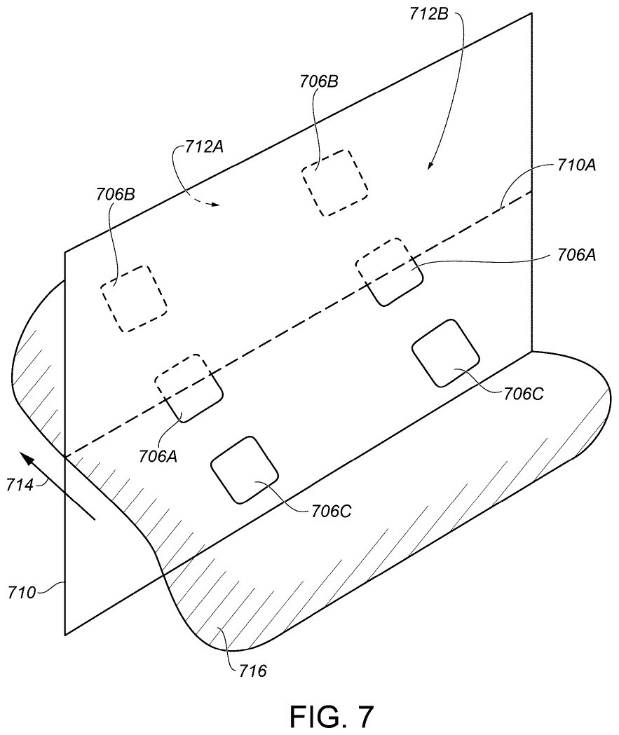

Embodiments of the present invention are not limited to cases in which the transducers of the first transducer set are arranged in a perimeter-like or surrounding manner around a region of space. Other transducer arrangements may be employed in other embodiments. For example, in some embodiments, the first transducer set includes at least two transducers of the plurality of transducers (e.g., transducers 220, 306, 406), the at least two transducers arranged at non-coincident locations intersected by a virtual plane. For instance, in FIG. 7, transducers 706A form at least part of the first transducer set and are intersected by a virtual plane 710 (i.e., as represented by broken line 710A which lies on virtual plane 710). According to some embodiments, a set of transducers 706B lies behind (in relation to the perspective view of FIG. 7) the virtual plane 710 (i.e., on a first side 712A of the virtual plane). The set of transducers 706B are depicted with broken lines (as are the back half of transducers 706A) to indicate that they reside behind the virtual plane 710 in the view presented in FIG. 7. According to some embodiments, a set of transducers 706C lies in front of the virtual plane 710 (i.e., on a second side 712B of the virtual plane) as seen in the view presented in FIG. 7. The transducers 706A, 706B, and 706C are shown located at least proximate to, or abutting a tissue surface 716 in FIG. 7, according to some embodiments. According to some embodiments, the data processing device system 110, 310 may be configured by the program at least to determine, for inclusion in the second transducer set, particular transducers of the plurality of transducers (e.g., transducers 220, 306, 406) as located at least in part or entirely on a first side of the virtual plane as satisfying the determined positional relationship with respect to the first transducer set. For example, according to some embodiments associated with FIG. 7, the set of transducers 706B (i.e., rather than the set of transducers 706C) may be determined as satisfying the determined positional relationship with respect to the transducers 706A selected as part of the first transducer set, and in some embodiments, the set of transducers 706C (i.e., rather than the set of transducers 706B) may be determined as satisfying the determined positional relationship with respect to the transducers 706A selected as part of the first transducer set. (It should be noted that, although this description typically refers to the first side of the virtual plane with reference 712A and the second side of the virtual plane with reference 712B, they may be flipped or opposite in some embodiments, such that the first side of the virtual plane may refer to reference 712B, and the second side of the virtual plane may refer to reference 712A.)

In some embodiments, the data processing device system 110, 310 may be configured by the program at least to determine the first side (e.g., 712A) of the virtual plane, the second side (e.g., 712B) of the virtual plane, or each of the first side and the second side based at least on user input received via the input-output device system 120, 320. In some embodiments, the data processing device system 110, 310 may be configured by the program at least to determine the first side (e.g., 712A) of the virtual plane, the second side (e.g., 712B) of the virtual plane, or each of the first side and the second side based at least on an analysis of transducer data. For example, in some embodiments, the transducer data may be provided by at least some of the plurality of transducers. Determining the first side (e.g., 712A) of the virtual plane, the second side (e.g., 712B) of the virtual plane, or each of the first side and the second side based at least on an analysis of transducer data may be accomplished in various ways according to various embodiments. For example, in some embodiments, the data processing device system 110, 310 may be configured by the program at least to determine, via an analysis of transducer data, a propagation of electrophysiological activity through the virtual plane (e.g., 710). In some embodiments, the data processing device system is configured by the program at least to determine the first side (e.g., 712A) as a downstream side of the virtual plane, the downstream side of the virtual plane determined in accordance with a direction (e.g., direction 714) of the propagation of the electrophysiological activity through the virtual plane. In some embodiments, the data processing device system 110, 310 may be configured by the program at least to determine, via an analysis of transducer data, a propagation of electrophysiological activity through the virtual plane (e.g., 710). In some embodiments, the data processing device system is configured by the program at least to determine the first side as an upstream side of the virtual plane, the upstream side of the virtual plane determined in accordance with a direction of the propagation of the electrophysiological activity through the virtual plane.

The transducer data employed to determine the propagation of the electrophysiological activity may be provided by at least some of the plurality of transducers (e.g., transducers 220, 306, 406) in some embodiments. For example, with reference to FIG. 7, electrophysiological activity may be detected by transducers 706B, 706C, and in some cases by transducers 706A when they are also configured to sense electrophysiological activity. Various voltage maps including activation or propagation maps may be determined from the spaced apart transducers 706B, 706C to determine the direction of propagation based on the timing of a sensed voltage wavefront crossing the spaced apart transducers 706B, 706C. Other methods may be employed to determine propagation direction according to various embodiments. For example, techniques related to omnipolar electrograms may identify a propagation direction based on which direction maximizes some property of the resulting omnipolar electrogram. (e.g. max peak-to-peak, correlation with time-derivative of electrograms, etc.). In some embodiments, the direction of propagation of the electrophysiological activity may be determined prior to an activation of the first transducer set including transducers 706A. Once the direction of propagation of the electrophysiological activity has been determined, the effectiveness of a lesion formed by the activation of transducer 706A in blocking the propagation of the electrophysiological activity may be assessed. According to various embodiments, propagation of the electrophysiological activity may be simulated by transducer stimulation according to some embodiments.

In some embodiments, the determination of the second transducer set is not made in response to, or directly in response to, the selection of the first transducer set. For example, referring to FIG. 5A, methods 500 may include block 502b associated with computer-executable instructions (e.g., activation instructions provided by a program) configured to cause a data processing device system (e.g., 110, 310) to cause activation of a first transducer set of a plurality of transducers (e.g., transducers 220, 306, 406) of at least a portion of a transducer-based device, the at least the portion of the transducer-based device positionable within a bodily cavity. In some embodiments, the first transducer set that is caused to be activated as per block 502b is or includes transducers selected for inclusion in the first transducer set selected as per block 502a. For instance, in some embodiments, block 502a represents a selection of the first transducer set for ablation activation, and block 502b represents the ablation activation of those transducers in the first transducer set selected per block 502a. In some embodiments, the particular transducer graphical elements 620 corresponding to the transducers activated as per block 502b may include a particular visual characteristic set indicative of the activation. For example, the use of the bolded outlined transducer graphical elements 620 described above in the context of transducer selection, according to various embodiments, may be used in the context of transducer activation in other embodiments. According to various embodiments, the activation of the first transducer set may cause each transducer of the first transducer set to transmit tissue ablative energy. According to various embodiments, each transducer in the first transducer set may be configured to transmit particular energy configured to cause pulsed field ablation of tissue. In some embodiments, each transducer in the first transducer set may be configured to transmit particular energy configured to cause thermal ablation of tissue. According to various embodiments, methods 500 may include block 504b associated with computer-executable instructions (e.g., determination instructions provided by a program) configured to cause a data processing device system (e.g., 110, 310) to determine, at least in response to the activation of the first transducer set, a second transducer set of the plurality of transducers (e.g., transducers 220, 306, 406) satisfying a determined positional relationship with respect to the first transducer set. According to various embodiments, each transducer in the second transducer set may be configured to record a respective electrogram. According to various embodiments, the second transducer set is mutually exclusive with the first transducer set. It is noted that various embodiments described above in this disclosure regarding the selection of a first transducer set according to block 502a may, in some embodiments, also be applied to the selection of the first transducer set for activation according to block 502b. It is noted that various embodiments described above in this disclosure regarding the determination of the second transducer set according to block 504a may, in some embodiments, also be applied to the determination of the second transducer set according to block 504b. It is noted that various embodiments described above in this disclosure regarding the determined positional relationship between the first and second transducer sets per block 504a may, in some embodiments, also be applied to the determined positional relationship between the first and second transducer sets per block 504b. In this regard, in some embodiments, blocks 504a and 504b may be the same or substantially the same (and therefore, the above discussed disclosures with respect to block 504a may also apply to block 504b) except that block 504b is executed in response to activation of a first transducer set per block 502b, and block 504a is executed in response to a selection of a first transducer set per block 502a, in some embodiments. In some embodiments, the activated first transducer set per block 502a is an activation of the first transducer set selected per block 502a.

In some embodiments, the positional relationship with respect to the first transducer set specified in block 504b may be determined prior to the activation of the first transducer set. In some embodiments, the positional relationship with respect to the first transducer set specified in block 504b is determined based at least on particular data stored in the memory device system, the particular data indicating a predetermined positional relationship with respect to at least an activated transducer set. In some embodiments, the first transducer set may include at least three transducers (e.g., at least those corresponding to transducer graphical elements 620C, 620D, and 620E in FIG. 6C) of the plurality of transducers, the at least three transducers arranged at non-colinear locations bounding (e.g., forming at least part of a boundary of) a region of space (e.g., corresponding to graphical region 636A) interior the non-colinear locations of the at least three transducers. The data processing device system 110, 310 may be configured by the program at least to determine, for inclusion in the second transducer set, particular transducers of the plurality of transducers located at least in part or entirely in the region of space (e.g., corresponding to graphical region 636A) as satisfying the determined positional relationship with respect to the first transducer set specified in block 504b, at least in some embodiments in which the determined positional relationship is, e.g., within a region (e.g., represented by graphical region 636A) interior (e.g., at least in part on a concave side) of the first transducer set. In some embodiments, each transducer in the second transducer set may be configured to transmit tissue ablative energy.

Referring to FIG. 5A, methods 500 may include block 506 associated with computer-executable instructions (e.g., activation instructions provided by a program) configured to cause a data processing device system (e.g., 110, 310) to cause, via the input-output device system, display of at least part of an electrogram set. For example, the second portion 600B of the graphical representation 600 of various ones of FIG. 6 shows a display of a set of electrocardiograms 650 and a set electrograms 660, the set of electrograms also referred to as the “electrogram set”, according to some embodiments or states of the user interface. According to some embodiments, the set of electrocardiograms 650 is produced by electrophysiological voltage data recorded by transducers placed externally on the body. According to various embodiments, the electrogram set 660 is produced by electrophysiological voltage data recorded internally within the body. For example, and as exemplified in various ones of FIGS. 6, the electrogram set 660 may be recorded by at least some of the transducers of the plurality of transducers (e.g., transducers 220, 306, 406), according to some embodiments. It is noted that the display of electrograms may occur at any time in methods 500 and need not be limited to the display of electrograms only in association with block 506, according to various embodiments. For instance, various electrograms may be continually displayed so long as electrograms are being sensed, in some embodiments. In this regard, in some embodiments, block 506 may be associated with a modification or supplementation of an existing display of electrograms, such as, e.g., in response to the determination of a second transducer set as per either or both of blocks 504a and 504b, as will be described in more detail below. It is noted that, in FIG. 5A, however, the display of electrograms as per block 506 need not occur in response to any action performed in either or both of blocks 504a and 504b, according to some embodiments.

In various ones of FIGS. 6, each electrogram of set of electrograms 660 corresponds to, or is recorded by, a respective transducer of the plurality of transducers (e.g., transducers 220, 306, 406), according to some embodiments. Accordingly, in some embodiments, each electrogram is identified with an alpha-numeric designator used to identify respective ones of the transducers and the respective transducer graphical element 620 (for example, described above in this disclosure). According to some embodiments associated with FIG. 6A, the electrograms 660 are presented consecutively in an ordered array starting with the “1” series (associated with radial line “1” per discussion above, i.e., “1A”, and proceeding with “1B”, “1C”, “ID” . . . ), then with the “2″series (associated with radial line “2” per discussion above, i.e., “2C”, “2D”, “2E” . . . ), then with the “3” series (associated with radial line “3” per discussion above, i.e., “3B”, “3C”, “3D” . . . ) and then with a portion of the “4” series (associated with radial line “4” per discussion above, i.e., “4C” and “4D”). The electrogram designated with alpha-numeric designator “1A”, for example, corresponds to the transducer graphical element 620 also associated with alpha-numeric designator “1A” (not shown in the orientation of the graphical representation of the transducer-based device in FIG. 6A, but shown in in FIG. 6B as transducer graphical element 620F).

According to various embodiments, other electrograms (e.g., from a remaining portion of the “4” series of electrograms, the “5” series of electrograms, the “6” series of electrograms, and continuing up to and including the “16” series of electrograms are not visually displayed since they number too many to be included in the visual displayed area of graphical representation 600. It is noted that, according to various embodiments, some of the electrograms that are not visually displayed, may, based on user input, for example, be visually displayed. For example, the ordered array of electrograms may be manipulated (e.g., scrolled by pressing of a directional keyboard key or rotation of a mouse wheel or a directional sliding of a finger on a touch screen, etc.) to visually display electrograms that were not displayed as shown at least with FIG. 6B, where the ordered array of electrograms 660 has been scrolled upwards to start at the set of electrograms 660 corresponding to electrode “5B”. It is noted that at least some of the electrograms that were visually displayed prior to such a manipulation, may not be visually displayed just after such manipulation. For example, in FIG. 6B (compared to the state of FIG. 6A), various electrograms in the “1” series, the “2” series, the “3” series, and the “4” series are no longer displayed after the scrolling manipulation, according to some embodiments.

As described above in this disclosure, FIGS. 6A and 6B visually display the selection or activation of the first transducer set (e.g., via the bolded outlined corresponding transducer graphical elements 620). According to various embodiments, in FIGS. 6A and 6B, the set of electrograms 660 does not visually indicate the selection of the first transducer set in any particular manner. In particular, none of the electrograms associated with any of the particular transducers of the first transducer set (e.g., whether selected via block 502a, or activated via block 502b) are rendered in a particular manner that visually accentuates or visually distinguishes them from the electrograms associated with particular transducers that do not form part of the first transducer set.

Referring to FIG. 5B, block 506 includes, according to various embodiments, various sub-blocks associated with computer-executable instructions (e.g., electrogram display instructions provided by the program). According to some embodiments, the various sub-blocks may include sub-block 506a associated with computer-executable instructions configured to cause the data processing device system (e.g., 110, 310) to cause, via the input-output device system, (a) a visual display of a first portion of the electrogram set in a state in which the first portion of the electrogram set was not visually displayed just prior to the visual display of the first portion of the electrogram set. Each electrogram in the first portion of the electrogram set may be derived from or correspond to, according to some embodiments, at least one electrogram recorded by at least one respective transducer in the second transducer set. According to some embodiments, the various sub-blocks of block 506 may include sub-block 506b associated with computer-executable instructions configured to cause the data processing device system (e.g., 110, 310) to alternatively or additionally cause, via the input-output device system 120, 320, (b) an increasing of a visual prominence of a second portion of the electrogram set in a state in which the second portion of the electrogram set was visually displayed just prior to the increasing of the visual prominence of the second portion of the electrogram set. Each electrogram in the second portion of the electrogram set may be derived from or correspond to, according to some embodiments, at least one electrogram recorded by at least one respective transducer in the second transducer set. In some embodiments, the data processing device system (e.g., 110, 310) is configured to cause, via the input-output device system, and as per both blocks 506a and 506b in FIG. 5B, both (a) and (b). In some embodiments, the data processing device system may be configured by the program at least to cause, via the input-output device system 120, 320, (a), (b), or (a) and (b) at least in response to the determination of the second transducer set (e.g., per either or both blocks 504a and 504b). In some embodiments, the data processing device system 110, 320 may be configured by the program at least to cause (a), (b), or (a) and (b) at least in response to initiation of activation of at least one transducer in the first transducer set as per block 502b.

For example, various thermal ablation modalities do not typically generate fields (e.g., electrics fields) that significantly adversely impact electrophysiological signals and, as a consequence, allow for meaningful (e.g., reasonably accurate) electrograms to be recorded. As such, in some embodiments, the display of meaningful electrogram information during the delivery of the thermal ablation energy is possible, along with the manipulation of the electrogram information in accordance with (a), (b), or (a) and (b).

In some embodiments, the data processing device system 110, 310 may be configured by the program at least to cause (a), (b), or (a) and (b) at least in response to completion of activation of at least one transducer in the first transducer set as per block 502b. For example, various pulsed field ablation modalities typically generate fields (e.g., electrics fields) that adversely impact or disrupt electrophysiological signals and, as a consequence, make it difficult to record meaningful (e.g., reasonably accurate) electrograms. As such, in some embodiments, the display of meaningful electrogram information generally occurs after the completion of the delivery of the pulsed field ablation, along with the manipulation of the electrogram information in accordance with (a), (b), or (a) and (b). In some embodiments, the display of meaningful electrogram information may occur during a treatment incorporating pulsed field ablation, for example, when the pulsed field ablation is provided by delivery of a plurality of pulse trains. In at least such a case, measurement or sensing of the electrogram signals from the bodily cavity may occur between adjacent pulse trains of the plurality of pulse trains in order to avoid the fields generated by the pulse trains that would adversely impact or disrupt the electrophysiological signals. Consequently, when measuring/sensing electrograms between adjacent pulse trains, reasonably accurate electrograms can be obtained.

According to some embodiments, an example of (a) is illustrated by the arrangement of electrograms 660 shown in the second portion 600B of the graphical representation 600 shown in FIG. 6C. In FIG. 6C, the electrograms 660 corresponding to the second set of transducers (e.g., transducers in a region corresponding to graphical region 636A and, e.g., determined via either block 504a or 504b, according to some embodiments) are identified as “8C”, “8D”, “8E”, “9B”, “9C”, “9D”, “9E”, “9F”, “10C”, “10D”, and “10E”. As compared with FIG. 6B, which corresponds to a state in which the second transducer set was not yet determined, FIG. 6C corresponds to the state in which a first portion 660A of the electrogram set is visually displayed in a state in which the first portion 660A of the electrogram set was not visually displayed just prior to the visual display of the first portion 660A of the electrogram set. In some embodiments associated with FIG. 6C, the first portion 660A of the electrogram set includes electrograms identified as “9B”, “9C”, “9D”, “9E”, “9F”, “10C”, “10D”, and “10E”, which, as per FIG. 6B, were not visually displayed just prior to the visual display of the first portion 660A of the electrogram set in FIG. 6C. According to various embodiments, each electrogram in the first portion 660A is derived from or corresponds to at least one electrogram recorded by at least one respective transducer in the second transducer set (e.g., electrogram identified as “9C” corresponds to transducer 9C, which is in the second transducer set, as indicated by the dotted outline of the corresponding transducer graphical element 620 in the state of FIG. 6C).

According to some embodiments, an example of (b) is illustrated by the arrangement of electrograms 660 shown in the second portion 600B of the graphical representation 600 shown in FIG. 6C. As stated above, in FIG. 6C, the electrograms 660 corresponding to the second set of transducers (e.g., transducers in a region corresponding to graphical region 636A and, e.g., determined via either block 504a or block 504b, according to some embodiments) are identified as “8C”, “8D”, “8E”, “9B”, “9C”, “9D”, “9E”, “9F”, “10C”, “10D”, and “10E”. In FIG. 6C an increase in the visual prominence of a second portion 660B of the electrogram set occurs in a state in which the second portion 660B of the electrogram state was visually displayed just prior to the increasing of the visual prominence of the second portion 660B of the electrogram set. In some embodiments associated with FIG. 6C, the second portion 660B of the electrogram set includes electrograms identified as “8C”, “8D”, and “8E”, which were also displayed in the state of FIG. 6B, but are displayed in the state of FIG. 6C with a greater visual prominence (e.g., are moved “up” or to or toward the beginning of the ordered array or list of electrograms) as compared with the state of FIG. 6B. Other manners of increasing visual prominence of an electrogram may be used, as discussed in more detail below.

According to various embodiments, the data processing device system 110, 310 may be configured by the program at least to cause (b) at least by causing, via the input-output device system 120, 320, each electrogram of the second portion 660B of the electrogram set to be displayed according to a first visual characteristic set just prior to the increasing of the visual prominence of the second portion 660B of the electrogram set, and cause, via the input-output device system 120, 320, each electrogram of the second portion 660B of the electrogram set to be displayed according to a second visual characteristic set upon the increasing of the visual prominence of the second portion of the electrogram set, the second visual characteristic set different than the first visual characteristic set. In this regard, various manners of increasing visual prominence of an electrogram may be implemented, such as, but not limited to, enhancing a display location (e.g., moving toward a center of a display region or providing a preferred ordering location) of the associated electrogram; bolding, highlighting, or enhancing a color, intensity, or brightness appearance of an aspect of the associated electrogram or its label; italicizing a label or changing to a more prominent font of a label for the associated electrogram; adding a graphical element or marker that visually calls attention to the associated electrogram; or decreasing the visual prominence of one or more other electrograms, which can have the effect of increasing the visual prominence of an electrogram that does not have its visual prominence decreased; according to various embodiments.

According to some embodiments, each electrogram of the second portion 660B of the electrogram set is displayed as a signal trace, each signal trace displayed with (i) a first graphical line type according to the first visual characteristic set, and (ii) a second graphical line type according to the second visual characteristic set, the second graphical line type more visually prominent than the first graphical line type. For example, in FIG. 6C, the signal trace of each electrogram 660 (i.e., “8C”, “8D, “8E”) of the second portion 660B of the electrogram set is shown with a “bolder” or “thicker” line type than the signal traces of the electrograms “8C”, “8D”, and “8E” shown in FIG. 6B. In some embodiments, the data processing device system 110, 310 may be configured by the program at least to cause (a) (e.g., described above with respect to block 506a) at least by causing, via the input-output device system 120, 320, each electrogram of the first portion of the electrogram set to be displayed according to the second visual characteristic set. For example, in FIG. 6C, the trace signals of the electrograms of the first portion 660A of the electrogram set (e.g., electrograms identified as “9B”, “9C”, “9D”, “9E”, “9F”, “10C”, “10D”, and “10E”) are also shown with “bolded” line forms in a similar manner as the second visual characteristic set governing the visual appearance of the electrograms of the second portion 660B of the electrogram set as per various embodiments.

According to some embodiments, the data processing device system 110, 310 may be configured by the program at least to cause, via the input-output device system 120, 320, a visual display of a third portion of the electrogram set just prior to the increasing of the visual prominence of the second portion of the electrogram set. For example, referring again to FIG. 6B, various electrograms 660 including electrograms in a third portion 660C of the electrogram set are shown just prior to the increasing of the visual prominence of the second portion 660B of the electrogram set shown in FIG. 6C. According to some embodiments, no electrogram in the third portion of the electrogram set is provided by any electrogram in the second portion of the electrogram sct. For example, with reference to FIGS. 6B and 6C, a third portion 660C. of the electrogram set may include, by way of example, at least the electrograms identified as “5G”, “5H”, “51”, and “6C”, none of which are included in the second portion 660B of the electrogram set, according to some embodiments. It is noted, that in some embodiments, that the clectrograms in the third portion 660C of the electrogram set need not be sequential electrograms as exemplified in the example above. According to some embodiments, each electrogram of the third portion 660C of the electrogram set is displayed according to a first visual characteristic set just prior to the increasing of the visual prominence of the second portion of the electrogram set. For example, in FIG. 6B in which the various electrograms 660 are shown just prior to the increasing of the visual prominence of the second portion 660B of the electrogram set as shown in FIG. 6C, the electrograms of the third portion 660C are displayed with signal traces having a solid continuous form. According to some embodiments, each electrogram of the third portion 660C of the electrogram set may be displayed according to a second visual characteristic set upon the increasing of the visual prominence of the second portion of the electrogram set, the second visual characteristic set less visually prominent than the first visual characteristic set. For example, as shown in FIG. 6C, upon increasing of the visual prominence of the second portion 660B of the electrogram set, the electrograms of the third portion 660C are shown with signal traces that have a “broken line” form to show a different set of visual characteristics as compared to their form in FIG. 6B. In this regard, in some embodiments, in addition to or as a way of increasing the visual prominence of at least the second portion 660B of the electrogram set, the visual prominence of various ones of other electrograms, such as those in the third portion 660C of the electrogram set, may be decreased. Accordingly, although not shown in FIG. 6C, all other electrograms besides those in the second portion 660B of the electrogram set may have their visual prominence decreased like the electrograms in the third portion 660C of the electrogram set, e.g., as a way to increase or further increase the visual prominence of the electrograms in the second portion 660B of the electrogram set between the states of FIGS. 6B and 6C, according to some embodiments. Although not shown in FIG. 6C, all other electrograms besides the electrograms of the electrogram set 660 corresponding to the second transducer set may have their visual prominence decreased like the electrograms in the third portion 660C of the electrogram set 660, e.g., as a way to increase or further increase the visual prominence of the electrograms of the electrogram set 660 corresponding to the second transducer set between the states of FIGS. 6B and 6C, according to some embodiments.

It is also noted that some electrogram traces in various ones of FIGS. 6, including the electrograms identified as “6G” and “6H” in FIG. 6C are illustrated as flat lines. Such a circumstance may occur, for instance, in a state in which the corresponding electrogram-sensing electrode is at too far of a distance from the tissue surface, thereby causing an insufficient detection of an electrogram. Such a circumstance may also occur when electrophysiological isolation has occurred after tissue ablation, such that cardiac electrical signals are no longer able to reach the corresponding electrogram-sensing electrode. In such a case, the flat-lined electrogram may act as a confirmation that tissue ablation has been successfully achieved.

According to various embodiments, each electrogram of the third portion 660C of the electrogram set may be displayed with (i) a first degree of opacity according to the first visual characteristic set, and (ii) a second degree of opacity according to the second visual characteristic set. According to various embodiments, the second degree of opacity may be less than the first degree of opacity. For example, although the signal traces of the electrograms of the third portion 660C of the electrogram set are shown in a broken line form in FIG. 6C to emulate such a reduced opacity, the contrast or intensity of the signal traces of the electrograms of the third portion 660C of the electrogram set may be reduced to provide the reduced opacity according to various embodiments. It is noted that, in FIG. 6C, the signal traces of the electrograms of the second portion 660B of the electrogram set have been bolded (i.e., as compared with their corresponding signal traces shown in FIG. 6B) to increase the visual prominence of the second portion 660B of the electrogram set according to various embodiments. In some embodiments however, during the implementation of (b) (block 506b in FIG. 5B), the visual characteristics of the electrograms of the second portion 660B of the electrogram set are not altered to increase the visual prominence of the second portion 660B of the electrogram set. Rather, in some embodiments, the visual prominence of particular visually displayed electrograms (e.g., the electrograms of the third portion 660C of the electrogram set) is reduced to accentuate the visual prominence of the second portion 660B of the electrogram set.

In some embodiments, at least one transducer in the first transducer set (e.g., reference at least blocks 502a, 502b, or both, in some embodiments) is configured to record an electrogram, and the third portion 660C of the electrogram set may include an electrogram recorded by the at least one transducer in the first transducer set. For example, in FIG. 6C, the third portion 660C of the electrogram set includes the electrogram identified as “6C” which was, according to some embodiments, recorded by a transducer in the first transducer set (e.g., as indicated by the corresponding bold outlined transducer graphical element 620 in at least FIG. 6C).

Methods other than changing the visual characteristics of each electrogram of the second portion of the electrogram set or each of other electrograms (e.g., as described above with respect to the third portion 660C of the electrogram set) may be employed according to various embodiments to increase the visual prominence of the second portion 660B of the electrogram set. For example, in some embodiments, the data processing device system 110, 310 may be configured by the program at least to cause, via the input-output device system 120, 320, a visual display of the electrogram set in an ordered array of electrograms, at least some of the electrograms in the second portion of the electrogram set separated from one another in the ordered array of electrograms by one or more electrograms not forming part of the second portion of the electrogram set; and cause (b) (referred to in block 506b) at least by reordering the electrograms in the ordered array of electrograms to form a reordered array of electrograms such that all the electrograms of the second portion of the electrogram set are arranged successively one after the other in the reordered array of electrograms without any interruption by any electrogram in the electrogram set not forming part of the second portion of the electrogram set. For example, consider a modification of FIG. 6C as an alternate example, wherein, in some embodiments, the second portion of the electrogram set may be second portion 660L and include the electrograms identified as “8C”, “8D”, “8E”, “9B”, “9C”, and “9D” (in contrast to second portion 660B). Considering such a modification, if the electrograms were to be visually displayed in an ordered array, the electrograms identified as “9B”, “9C”, and “9D” would be separated from the electrograms identified as “8C”, “8D”, and “8E” by a particular set of consecutive electrograms, such as electrograms identified as “8F”, “8G”, “8H”, “8I” and “9A”, with none of these electrograms in the particular set being recorded by any transducer in the second transducer set. By clustering the electrograms identified as “8C”, “8D”, and “8E” and the electrograms identified as “9B”, “9C” and “9D” without any intervening electrograms (e.g., “8F”, “8G”, “8H”, “81” and “9A”) therebetween as shown by second portion 660L in FIG. 6C, the clustered arrangement imparts a greater visual prominence to the second portion of the electrogram set. In other words, it may be considered that increasing a visual prominence of an electrogram set may include grouping together the electrograms of such electrogram set from a previous or immediately preceding state in which they were not grouped or were grouped in a different manner.

In some embodiments, at least one transducer in the first transducer set is configured to record an electrogram, and the one or more electrograms not forming part of the second portion of the electrogram set includes an electrogram recorded by the at least one transducer in the first transducer set. For example, an electrogram recorded by the transducer identified as “9A” would be an electrogram recorded by a transducer in the first transducer set (e.g., as indicated by the bold outline of the corresponding transducer graphical element 620), and such electrogram is not in the second portion (e.g., second portion 660L in this example) of the electrogram set identified as “8C”, “8D”, “8E”, “9B”, “9C”, and “9D”. In some embodiments, the data processing device system 110, 310 may be configured by the program at least to cause (b) (block 506b in FIG. 5B) at least by causing, via the input-output device system 120, 320, the reordering of the electrograms in the ordered array of electrograms to form the reordered array of electrograms such that all of the electrograms of the second portion of the electrogram set (e.g., second portion 660L in this example) appear at a beginning of the reordered array of electrograms. For example, in FIG. 6C, the clustered arrangement of the electrograms identified as “8C”, “8D”, and “8E” and the electrograms identified as 9B″, “9C” and “9D” that form the second portion (e.g., second portion 660L in this example) of the electrogram set appear, not only together as a group, in some embodiments, but may appear at the beginning of the displayed reordered array of electrograms 660 as shown.

In some embodiments, the data processing device system 110, 310 may be configured by the program at least to cause (a) (i.e., (a) referred to in block 506a) and (b) (i.e., (b) referred to in block 506b) at least by causing a visual display, via the input-output device system 120, 320, of the first portion (e.g., first portion 660A) of the electrogram set and the second portion (e.g., returning to the interpretation of FIG. 6C where the second portion of the electrogram set is second portion 660B) of the electrogram set successively one after the other in an ordered array of electrograms without any interruption by any electrogram in the electrogram set not forming part of a group of electrograms consisting of the first portion of the electrogram set and the second portion of the electrogram set. For example, in FIG. 6C, the electrograms identified as “8C”, “8D”, and “8E” of the second portion 660B of the electrogram set and the electrograms identified as “9B”, “9C”, “9D”, “9E”, “9F”, “10C”, “10D”, and “10E” of the first portion 660A of the electrogram set are arranged successively one after the other without any interruption by any electrogram that is not part of the group of electrograms identified as “8C”, “8D”, “8E”, “9B”, “9C”, “9D”, “9E”, “9F”, “10C”, “10D”, and “10E” (such group corresponding to the second transducer set associated with region 636A at least in the example of FIG. 6C). By clustering electrograms associated with the first portion 660A and the second portion 660B of the electrogram set, all or a greater number of electrograms in the determined second transducer set (e.g., determined per block 504a or block 504b and shown in FIG. 6C with dotted-outlined transducer graphical elements 620) can be visually displayed or more prominently visually displayed, thereby providing the user with improved efficiency of viewing potentially more relevant electrograms during a procedure.

According to some embodiments, e.g., associated with at least (a) described above with reference to block 506a, the data processing device system 110, 310 may be configured by the program at least to cause, via the input-output device system 120, 320, a visual display of a third portion of the electrogram set just prior to the visual display of the first portion of the electrogram set. According to various embodiments, each electrogram in the third portion of the electrogram set is derived from or corresponds to at least one electrogram recorded by at least one respective transducer in a third transducer set of the plurality of transducers (e.g., 220, 306, 406). According to various embodiments, the third transducer set does not include any transducer in the second transducer set. According to some embodiments, the data processing device system 110, 310 may be configured by the program at least to remove visual display, via the input-output device system 110, 310 and in response to at least the visual display of the first portion of the electrogram set in the state in which the first portion of the electrogram set was not visually displayed just prior to the visual display of the first portion of the electrogram set, of at least one electrogram in the third portion of the electrogram set that was displayed just prior to the visual display of the first portion of the electrogram set. For example, in FIG. 6B, a third portion 660D of the electrogram set includes electrograms identified as “6I”, “7B”, and “7C” and is shown in the state (of FIG. 6B) in which the first portion 660A of the electrogram set was not visually displayed just prior to the visual display (in the state of FIG. 6C) of the first portion 660A of the electrogram set. In this regard, FIG. 6C corresponds to the visual display of the first portion 660A of the electrogram set in the state in which the first portion 660A of the electrogram set was not visually displayed just prior (e.g., the state of FIG. 6B) to the visual display of the first portion 660A of the electrogram set. In the state of FIG. 6C, the visual display of the third portion 660D of the electrogram set has been removed, according to various embodiments. In some embodiments, at least one transducer in the first transducer set is configured to record an electrogram, and the third transducer set may include the at least one transducer in the first transducer set. For example, according to various embodiments, the third portion 660D of the electrogram set includes an electrogram identified as “7B”, which was recorded by a respective transducer of the plurality of transducers that was included in the first transducer set (as indicated by the bold outline of the corresponding transducer graphical element 620) and which also forms part of the third transducer set (corresponding to the third portion 660D of the electrogram set).

In some embodiments, the data processing device system 110, 310 may be configured by the program at least to cause, via the input-output device system 120, 320, a visual display of the electrogram set in an ordered array of electrograms (for example as shown at least in FIG. 6B). According to various embodiments, the data processing device system 110, 310 may be configured by the program at least to cause, via the input-output device system 120, 320, a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers. In some embodiments, the third transducer set does not include any transducer in the second transducer set. In some embodiments, the data processing device system 110, 310 may be configured by the program at least to cause, via the input-output device system 120, 320, visual repositioning, in response to at least the visual display of the first portion of the electrogram set in the state in which the first portion of the electrogram set was not visually displayed just prior to the visual display of the first portion of the electrogram set, of at least some of the electrograms in the third portion of the electrogram set in the ordered array of electrograms. For example, as compared between FIGS. 6B and 6C, particular electrograms of the third portion 660C of the electrogram set (i.e., electrograms identified as “5G”, “5H”, “51”, and “6C”) are visually repositioned in response to at least the visual display of the first portion 660A of the electrogram set in the state in which the first portion 660A of the electrogram set was not visually displayed just prior to the visual display of the first portion 660A of the electrogram set. In some embodiments, at least one transducer in the first transducer set is configured to record an electrogram, and the third transducer set includes the at least one transducer in the first transducer set. For example, the third portion 660C of the electrogram set includes electrogram 6C which was recorded by a transducer in the first transducer set (as indicated by the bold outline of the corresponding transducer graphical element 620) and which also forms part of the third transducer set 660C.

In some embodiments, e.g., associated with (a) (referenced in block 506 (a) in FIG. 5B), the data processing device system 110, 310 may be configured by the program at least to cause, via the input-output device system 120, 320, a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers. According to some embodiments, the third transducer set does not include any transducer in the second transducer set. In some embodiments, the data processing device system 110, 310 may be configured by the program at least to cause, via the input-output device system 120, 320, visual display, in response to at least the visual display of the first portion of the electrogram set in the state in which the first portion of the electrogram set was not visually displayed just prior to the visual display of the first portion of the electrogram set, of each electrogram of the first portion of the electrogram set with a greater visual prominence than the visual display of each of at least some of the electrograms in the third portion of the electrogram set. For example, in FIG. 6C, the third portion 660C of the electrogram set may include, by way of example, at least the electrograms identified as “5G”, “5H”, “51”, and “6C”, none of which are included in the second portion 660B of the electrogram set, according to some embodiments. FIG. 6C corresponds to at least the visual display of the first portion 660A of the electrogram set (e.g., electrograms identified as “9B”, “9C”, “9D”, “9E”, “9F”, “10C”, “10D”, and “10E”) in the state in which the first portion 660A of the electrogram set was not visually displayed just prior (FIG. 6B) to the visual display of the first portion 660A of the electrogram set. According to some embodiments, each electrogram of the first portion 660A of the electrogram set has a greater visual prominence than the visual display of each of at least some of the electrograms in the third portion 660C of the electrogram set shown in FIG. 6C (e.g., the signal traces of the electrograms of the first portion 660A are shown with a “bolded” line form, whereas the signal traces of the electrograms of the third portion 660C are shown with a “broken” line form). In some embodiments, at least one transducer in the first transducer set is configured to record an electrogram, and the at least some of the transducers in the third transducer set include the at least one transducer in the first transducer set. For example, in FIG. 6C, the third portion 660C of the electrogram set includes an electrogram identified as “6C”, which was recorded by a transducer in the first transducer set (as indicated by the bold outline of the corresponding transducer graphical element 620), and which forms part of the third transducer set (corresponding to the third portion 660C of the electrogram set).

According to various embodiments, the first transducer set in FIG. 6C (e.g., as graphically exemplified via their respective transducer graphical elements 620 whose outlines have been bolded), when activated, is configured or arranged in a ring-like manner to create a circumferentially arranged or ring-like tissue ablation zone. According to various embodiments, the determined second transducer set (e.g., determined per block 504a or 504b in FIG. 5A) includes particular transducers determined to be inside of the ring-like arrangement of the transducers of the first transducer set, as shown in FIG. 6C, where the dotted-outlined transducer graphical elements 620 correspond to the determined second transducer set, in some embodiments. According to various embodiments, increasing the visual prominence (e.g., via (a), (b), or (a) and (b) described above with respect to blocks 506a, 506b in FIG. 5B) of the respective electrograms 660 of the transducers of the determined second transducer set advantageously allows a user (e.g., health care practitioner) to readily determine when electrophysiological isolation (e.g., the signal traces will substantially flat-line) has occurred during the tissue ablation activation of the first transducer set, thereby allowing the user to assess when the completion of a transmural tissue lesion has occurred or to assess the efficacy of the formed lesion.

Referring to FIG. 6D, the first transducer set (e.g., as graphically exemplified via their respective transducer graphical elements 620 whose outlines have been bolded), when activated, is configured or arranged in a ring-like manner to create a circumferentially arranged or ring-like tissue ablation zone. According to FIG. 6D, the transducer graphical elements 620 identified as “IA”, “3B”, “5B”, “5C”, “5D”, “5E”, “5F”, “6F”, “6G”, “6H”, “7H”, “8H”, “81”, “91”, “101”, “11G”, “11H”, “11I”, “12F”, “12G”, “13C”, “13D”, “13E”, “13F”, “14C”, and “15B” correspond to the transducers of the plurality of transducers (e.g., transducers 220, 306, 406) that have been determined (e.g., per block 504a or 504b in FIG. 5A) to form the second transducer set, according to various embodiments. In FIG. 6D, according to some embodiments, the transducers determined for inclusion in the second transducer set (e.g., via blocks 504a or 504b) are outside the ring-like arrangement of the transducers of the first transducer set. According to various embodiments associated with FIG. 6D, the second portion 600B of the graphical representation 600 includes an arrangement 660E of electrograms, each electrogram in the arrangement 660E corresponding to a respective transducer of the determined second transducer set. In some embodiments, only the electrograms recorded by the transducers of the determined second transducer set are shown, while other particular electrograms (for example, electrograms recorded by the first transducer set, or other transducer sets, such as the third transducer sets described above) are not shown. According to various embodiments, a machine-based or automatic clustering of the electrograms of interest (e.g., the electrograms associated with the second transducer set) in this manner may be advantageously used to increase the visual prominence of the electrograms of interest without possible visual distractions that may arise from the presence of other electrograms. It is noted that such embodiments may also take place with a second transducer set arranged differently than exemplified in FIG. 6D. According to various embodiments, increasing the visual prominence (e.g., via (a), (b), or (a) and (b) described above) of the respective electrograms 660 of the transducers of the determined second transducer set arranged outwardly or outside of the first transducer set as exemplified in FIG. 6D advantageously allows a positive control for artificial stimulation (e.g., a pacing signal emitted by a transducer) and thereby serves as a timing reference for evaluation of the internal transducer set.

FIG. 6E shows graphical representation 600, according to some embodiments. In a manner similar to, or the same as FIG. 6B, transducer graphical elements 620 corresponding to the first transducer set (e.g., selected as per block 502a or activated as per block 502b) are shown in “bolded” (single line) outlines (in contrast to “bolded” double line outlined transducers 5D and 11G discussed below) and include the transducer graphical elements identified as “6C”, “6D”, “6E”, “7B”, “7C”, “7D”, “7E”, “7F”, “7G”, “8F”, “8G”, “9A”, “9G”, “9H”, “10F”, “10G”, “10H”, “11B”, “11C”, “11D”, “11E”, “11F”, “12C”, “12D”, “12E”, and “13B” according to some embodiments. According to various embodiments like that shown in FIG. 6E, the transducers of the first transducer set are arranged in a ring-like arrangement. According to some embodiments, a first particular second transducer set is determined by the data processing device system 110, 330 (e.g., according to the instructions associated with block 504a or block 504b), the transducers of the first particular second transducer set satisfying a first determined positional relationship with respect to the first transducer set in which transducers inside or interior of the ring-like arrangement of the first transducer set are determined for inclusion in the first particular second transducer set. In a manner similar to, or the same as FIG. 6B, transducer graphical elements 620 corresponding to the first particular second transducer set are shown with “dotted” outlines and include the transducers associated with transducer graphical elements identified as “8C”, “8D”, “8E”, “9B”, “9C”, “9D”, “9E”, “9F”, “10C”, “10D”, and “10E”, according to some embodiments. According to some embodiments, a second particular second transducer set is determined by the data processing device system 110, 330 (e.g., according to the instructions associated with block 504a or 504b), the transducers of the second particular second transducer set satisfying a second determined positional relationship with respect to the first transducer set in which transducers outside or exterior of the ring-like arrangement of the first transducer set are determined for inclusion in the second particular second transducer set. According to some embodiments, transducer graphical elements 620 corresponding to the second particular second transducer set are shown with “double-lined” outlines and include the transducer graphical elements transducers associated with transducer graphical elements identified as “5D”, and “11G” according to some embodiments. Although FIG. 6E only shows two transducer-graphical elements “5D” and “11G” as associated with transducers determined to be included in the second particular second transducer set for case of illustration, fewer or more transducers may be included, e.g., depending on the applicable determined positional relationship(s). For instance, the determined positional relationship might be not only transducers outside or exterior of the ring-like arrangement of electrodes in the first transducer set, but also may require adjacency with a transducer in the first transducer set. According to various embodiments, the instructions associated with blocks 504a and 504b may be associated with the determination of multiple second transducer sets, each second transducer set associated with a particular positional relationship with the first transducer set.

According to various embodiments, the electrograms 660 in the second portion 600B of the graphical representation 600 are displayed according to the instructions of block 506. According to various embodiments, the electrograms 660 have been reordered from the arrangement shown in FIG. 6B to the arrangement shown in FIG. 6E. According to various embodiments, the electrograms 660 recorded by the transducers of the first particular second transducer set (corresponding to the dotted-outline transducer graphical elements 620 in FIG. 6E) are manipulated into electrogram arrangement 660F, while the electrograms 660 recorded by the transducers of the second particular second transducer set (corresponding to the double-lined outlined transducer graphical elements 620 in FIG. 6E) are manipulated into electrogram arrangement 660G in FIG. 6E. Various electrograms 660 associated with transducers other than those associated with the first particular second transducer set and the second particular second transducer set are shown in the electrogram arrangement 660H in FIG. 6E, according to various embodiments. According to various embodiments, the electrograms associated with the first particular second transducer set are manipulated so as to be clustered or grouped together in the electrogram arrangement 660F, the electrograms associated with the second particular second transducer set are manipulated so as to be clustered or grouped together in the electrogram arrangement 660G. Each of the electrogram arrangement 660F, electrogram arrangement 660G, and electrogram arrangement 660H are spatially separated from one another in the reordered array of electrograms 660, thereby increasing the visual prominence of each of at least the electrogram arrangement 660F and the electrogram arrangement 660G. Other methods may be additionally or alternatively be employed to increase the visual prominence of each of at least the electrogram arrangement 660F and electrogram arrangement 660G. For example, in FIG. 6E, the signal traces of the electrograms in the electrogram arrangement 660F are shown with a relatively thick or bold line type, the signal traces of the electrograms in the electrogram arrangement 660G are shown in a relatively thin line type, and the signal traces of the electrograms in the electrogram arrangement 660H are shown in a broken line type by way of a non-limiting example.

It is noted that the manipulation of various ones of the electrograms displayed in FIG. 6B may be processed according to (a), (b), or (a) and (b) as described above in this disclosure with respect to blocks 506a and 506b in FIG. 5B to provide the various electrogram arrangements shown in FIG. 6E. Advantageously, a user (e.g., a health care provider) may readily distinguish the electrograms associated with the first particular second transducer set from the electrograms associated with the second particular second transducer set according to some embodiments. In some embodiments, a user (e.g., a health care provider) may readily distinguish the electrograms associated with each of the first particular second transducer from other electrograms not associated with either of the first particular second transducer set or the second particular transducer set. According to various embodiments, increasing the visual prominence (e.g., via (a), (b), or (a) and (b) described above with respect to blocks 506a, 506b) of the respective electrograms 660 of the transducers of the determined first particular second transducer set (whose corresponding transducer graphical elements 620 are shown in dotted outline in FIG. 6E) advantageously allows a user (e.g., health care practitioner) to readily determine when electrophysiological isolation has occurred during the tissue ablation activation of the first transducer set, thereby allowing the user to promptly and efficiently assess when the completion of a transmural tissue lesion has occurred or to promptly and efficiently assess the efficacy of the formed lesion. According to various embodiments, the increased visual prominence of the electrograms associated with the second particular second transducer set also provides benefits. For example, while the user is assessing whether the electrograms associated with the first particular second transducer set have indicated that electrophysiological isolation has been achieved (e.g., their signal traces will substantially “flat-line”), the presence of electrophysiological activity just outside of the formed circumferential lesion may be used to confirm that any isolation indicated by the electrograms associated with the first particular second transducer set is indeed valid due to the presence of electrograms associated with the second particular second transducer set confirming that cardiac electrical signals are indeed present outside the formed circumferential lesion. All of this information is readily available to the user in an unambiguous format according to the various embodiments described herein. In a similar manner, the embodiments described above with respect to FIG. 7 may be employed to determine the difference between the electrophysiological activity upstream and downstream of the transducers 706A to in turn determine whether an effective electrophysiological conduction block has been created by the ablation activation of transducers 706A.

With reference to FIG. 5B for example, according to various embodiments, at least one electrogram in the first portion of the electrogram set is a unipolar electrogram, at least one electrogram in the second portion of the electrogram set is a unipolar electrogram, or at least one electrogram in the first portion of the electrogram set and at least one electrogram in the second portion of the electrogram set is a unipolar electrogram. For example, in FIGS. 6, each of the electrograms 660 is a unipolar electrogram, each unipolar electrogram recorded by a respective single transducer. In some embodiments, (i) at least one electrogram in the first portion of the electrogram set is a bipolar electrogram, the bipolar electrogram derived from two electrograms recorded by two respective transducers in the second transducer set, (ii) at least one electrogram in the second portion of the electrogram set is a bipolar electrogram, the bipolar electrogram derived from two electrograms recorded by two respective transducers in the second transducer sct, or (i) and (ii). Bipolar electrograms are typically derived from signals recorded by a pair of neighboring transducers and provide localized measurements of the electrophysiological activity. Although bipolar electrograms may typically be less susceptible to low-frequency noise (e.g., as compared with unipolar electrograms), bipolar electrograms are greatly affected by the direction of wavefront propagation relative to the neighboring transducer pair. According to various transducers, (1) at least one electrogram in the first portion of the electrogram set is an omnipolar electrogram, the omnipolar electrogram derived from multiple electrograms recorded by multiple respective transducers in the second transducer set, (2) at least one electrogram in the second portion of the electrogram set is an omnipolar electrogram, the omnipolar electrogram derived from multiple electrograms recorded by multiple respective transducers in the second transducer set, or (1) and (2). An omnipolar electrogram is a form of bipolar electrogram computed from nearby but differently oriented pairs or neighboring or adjacent transducers. Accordingly, omnipolar electrograms typically are not adversely affected by or directionally dependent on local direction of the wavefront of the electrophysiological activity.

While some of the embodiments disclosed above are described with examples of cardiac electrophysiological activity, the same or similar embodiments may be used in other bodily organs, for example, the brain.

While some of the embodiments disclosed above are described with examples of cardiac ablation, the same or similar embodiments may be used for ablating other bodily organs or any lumen or cavity into which the devices of the present invention may be introduced.

Subsets or combinations of various embodiments described above can provide further embodiments.

These and other changes can be made to the invention in light of the above-detailed description. In general, in the following claims, the terms used should not be construed to limit the invention to the specific embodiments disclosed in the specification and the claims but should be construed to include other transducer-based device systems including all medical treatment device systems and all medical diagnostic device systems in accordance with the claims. Accordingly, the invention is not limited by the disclosure, but instead its scope is to be determined entirely by the following claims.

What is claimed is:

1. A medical system comprising:
a data processing device system;
an input-output device system communicatively connected to the data processing device system; and
a memory device system communicatively connected to the data processing device system and storing a program executable by the data processing device system, the data processing device system configured by the program at least to:
receive input via the input-output device system indicating a selection of a first transducer set of a plurality of transducers of at least a portion of a transducer-based device, the at least the portion of the transducer-based device positionable within a bodily cavity, each transducer in the first transducer set configured to transmit tissue ablative energy;
determine, at least in response to the selection of the first transducer set, a second transducer set of the plurality of transducers satisfying a determined positional relationship with respect to the first transducer set, each transducer in the second transducer set configured to record a respective electrogram, and the second transducer set mutually exclusive with the first transducer set;
cause, via the input-output device system, display of at least part of an electrogram set; and
cause, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

2. The medical system of claim 1, wherein each transducer in the second transducer set is configured to transmit tissue ablative energy.

3. The medical system of claim 1, wherein each transducer in the first transducer set is configured to record a respective electrogram.

4. The medical system of claim 1, wherein each transducer in the first transducer set and each transducer in the second transducer set comprises an electrode.

5. The medical system of claim 1, wherein each transducer in the first transducer set is configured to transmit particular energy configured to cause pulsed field ablation.

6. The medical system of claim 1, wherein each transducer in the first transducer set is configured to transmit particular energy configured to cause thermal ablation.

7. The medical system of claim 1, wherein the positional relationship with respect to the first transducer set is determined prior to the selection of the first transducer set.

8. The medical system of claim 1, wherein the positional relationship with respect to the first transducer set is determined based at least on particular data stored in the memory device system, the particular data indicating a predetermined positional relationship with respect to at least a selected transducer set.

9. The medical system of claim 1, wherein the first transducer set comprises at least three transducers of the plurality of transducers, the at least three transducers arranged at non-colinear locations bounding a region of space interior the non-colinear locations of the at least three transducers, and wherein the data processing device system is configured by the program at least to determine, for inclusion in the second transducer set, particular transducers of the plurality of transducers located at least in part or entirely in the region of space as satisfying the determined positional relationship with respect to the first transducer set.

10. The medical system of claim 9, wherein a location of each transducer in the first transducer set is arranged at a respective location, the respective locations of at least some of the transducers in the first transducer set bounding the region of space.

11. The medical system of claim 9, wherein various transducers in the first transducer set are distributed to at least partially surround a particular anatomical feature in the bodily cavity.

12. The medical system of claim 11, wherein the bodily cavity is a cardiac cavity, and the particular anatomical feature is a bodily opening in the cardiac cavity.

13. The medical system of claim 1, wherein the first transducer set comprises at least three transducers of the plurality of transducers, the at least three transducers arranged at non-colinear locations bounding a region of space exterior the non-colinear locations of the at least three transducers, and wherein the data processing device system is configured by the program at least to determine, for inclusion in the second transducer set, particular transducers of the plurality of transducers located at least in part or entirely in the region of space as satisfying the determined positional relationship with respect to the first transducer set.

14. The medical system of claim 13, wherein the location of each transducer in the first transducer set is arranged at a respective location, the respective locations of the transducers in the first transducer set bounding the region of space.

15. The medical system of claim 13, wherein various transducers in the first transducer set are distributed to at least partially surround a particular anatomical feature in the bodily cavity.

16. The medical system of claim 15, wherein the bodily cavity is a cardiac cavity, and the particular anatomical feature is a bodily opening in the cardiac cavity.

17. The medical system of claim 1, wherein:
the first transducer set comprises at least two transducers of the plurality of transducers, the at least two transducers arranged at non-coincident locations intersected by a virtual plane, and
the data processing device system is configured by the program at least to determine, for inclusion in the second transducer set, particular transducers of the plurality of transducers as located at least in part or entirely on a first side of the virtual plane as satisfying the determined positional relationship with respect to the first transducer set.

18. The medical system of claim 17, wherein the data processing device system is configured by the program at least to determine the first side, a second side of the virtual plane, or each of the first side and the second side based at least on user input received via the input-output device system.

19. The medical system of claim 17, wherein the data processing device system is configured by the program at least to determine the first side, a second side of the virtual plane, or each of the first side and the second side based at least on an analysis of transducer data.

20. The medical system of claim 19, wherein the transducer data is provided by at least some of the plurality of transducers.

21. The medical system of claim 17, wherein the data processing device system is configured by the program at least to determine, via an analysis of transducer data, a propagation of electrophysiological activity through the virtual plane, the data processing device system further configured by the program at least to determine the first side as a downstream side of the virtual plane, the downstream side of the virtual plane determined in accordance with a direction of the propagation of the electrophysiological activity through the virtual plane.

22. The medical system of claim 21, wherein the transducer data is provided by at least some of the plurality of transducers.

23. The medical system of claim 17, wherein the data processing device system is configured by the program at least to determine, via an analysis of transducer data, a propagation of electrophysiological activity through the virtual plane, the data processing device system further configured by the program at least to determine the first side as an upstream side of the virtual plane, the upstream side of the virtual plane determined in accordance with a direction of the propagation of the electrophysiological activity through the virtual plane.

24. The medical system of claim 23, wherein the transducer data is provided by at least some of the plurality of transducers.

25. The medical system of claim 1, wherein the received input includes user-based input indicating the selection of the first transducer set.

26. The medical system of claim 1, wherein the received input includes machine-based input indicating the selection of the first transducer set.

27. The medical system of claim 1, wherein (c) at least one electrogram in the first portion of the electrogram set is a unipolar electrogram, (d) at least one electrogram in the second portion of the electrogram set is a unipolar electrogram, or (c) and (d).

28. The medical system of claim 1, wherein (c) at least one electrogram in the first portion of the electrogram set is a bipolar electrogram, the bipolar electrogram derived from two electrograms recorded by two respective transducers in the second transducer set, (d) at least one electrogram in the second portion of the electrogram set is a bipolar electrogram, the bipolar electrogram derived from two electrograms recorded by two respective transducers in the second transducer set, or (c) and (d).

29. The medical system of claim 1, wherein (c) at least one electrogram in the first portion of the electrogram set is an omnipolar electrogram, the omnipolar electrogram derived from multiple electrograms recorded by multiple respective transducers in the second transducer set, (d) at least one electrogram in the second portion of the electrogram set is an omnipolar electrogram, the omnipolar electrogram derived from multiple electrograms recorded by multiple respective transducers in the second transducer set, or (c) and (d).

30. The medical system of claim 1, wherein the data processing device system is configured by the program at least to cause, via the input-output device system, a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set, and wherein the data processing device system is configured by the program at least to remove visual display, via the input-output device system and in response to at least the visual display of the first portion of the electrogram set in the state in which the first portion of the electrogram set was not visually displayed just prior to the visual display of the first portion of the electrogram set, of at least one electrogram in the third portion of the electrogram set that was displayed just prior to the visual display of the first portion of the electrogram set.

31. The medical system of claim 30, wherein at least one transducer in the first transducer set is configured to record an electrogram, and the third transducer set includes the at least one transducer in the first transducer set.

32. The medical system of claim 1, wherein:
the data processing device system is configured by the program at least to cause, via the input-output device system:
a visual display of the electrogram set in an ordered array of electrograms;
a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set; and
visual repositioning, in response to at least the visual display of the first portion of the electrogram set in the state in which the first portion of the electrogram set was not visually displayed just prior to the visual display of the first portion of the electrogram set, of at least some of the electrograms in the third portion of the electrogram set in the ordered array of electrograms.

33. The medical system of claim 32, wherein at least one transducer in the first transducer set is configured to record an electrogram, and wherein the third transducer set includes the at least one transducer in the first transducer set.

34. The medical system of claim 1, wherein:
the data processing device system is configured by the program at least to cause, via the input-output device system:
a visual display of a third portion of the electrogram set just prior to the visual display of the first portion of the electrogram set, each electrogram in the third portion of the electrogram set derived from or corresponding to at least one electrogram recorded by at least one respective transducer in a third transducer set of the plurality of transducers, the third transducer set not including any transducer in the second transducer set; and
visual display, in response to at least the visual display of the first portion of the electrogram set in the state in which the first portion of the electrogram set was not visually displayed just prior to the visual display of the first portion of the electrogram set, of each electrogram of the first portion of the electrogram set with a greater visual prominence than the visual display of each of at least some of the electrograms in the third portion of the electrogram set.

35. The medical system of claim 34, wherein at least one transducer in the first transducer set is configured to record an electrogram, and wherein the at least some of the transducers in the third transducer set include the at least one transducer in the first transducer set.

36. The medical system of claim 1, wherein:
the data processing device system is configured by the program at least to:
cause, via the input-output device system, a visual display of the electrogram set in an ordered array of electrograms, at least some of the electrograms in the second portion of the electrogram set separated from one another in the ordered array of electrograms by one or more electrograms not forming part of the second portion of the electrogram set; and
cause (b) at least by reordering the electrograms in the ordered array of electrograms to form a reordered array of electrograms such that all the electrograms of the second portion of the electrogram set are arranged successively one after the other in the reordered array of electrograms without any interruption by any electrogram in the electrogram set not forming part of the second portion of the electrogram set.

37. The medical system of claim 36, wherein at least one transducer in the first transducer set is configured to record an electrogram, and the one or more electrograms not forming part of the second portion of the electrogram set includes an electrogram recorded by the at least one transducer in the first transducer set.

38. The medical system of claim 36, wherein the data processing device system is configured by the program at least to cause (b) at least by causing, via the input-output device system, the reordering of the electrograms in the ordered array of electrograms to form the reordered array of electrograms such that all of the electrograms of the second portion of the electrogram set appear at a beginning of the reordered array of electrograms.

39. The medical system of claim 1, wherein the data processing device system is configured by the program at least to cause (a) and (b) at least by causing a visual display, via the input-output device system, of the first portion of the electrogram set and the second portion of the electrogram set successively one after the other in an ordered array of electrograms without any interruption by any electrogram in the electrogram set not forming part of a group of electrograms consisting of the first portion of the electrogram set and the second portion of the electrogram set.

40. The medical system of claim 1, wherein the data processing device system is configured by the program at least to cause (b) at least by causing, via the input-output device system, each electrogram of the second portion of the electrogram set to be displayed according to a first visual characteristic set just prior to the increasing of the visual prominence of the second portion of the electrogram set, and cause via the input-output device system, each electrogram of the second portion of the electrogram set to be displayed according to a second visual characteristic set upon the increasing of the visual prominence of the second portion of the electrogram set, the second visual characteristic set different than the first visual characteristic set.

41. The medical system of claim 40, wherein the data processing device system is configured by the program at least to cause (a) at least by causing, via the input-output device system, each electrogram of the first portion of the electrogram set to be displayed according to the second visual characteristic set.

42. The medical system of claim 40, wherein each electrogram of the second portion of the electrogram set is displayed as a signal trace, each signal trace displayed with (i) a first graphical line type according to the first visual characteristic set, and (ii) a second graphical line type according to the second visual characteristic set, the second graphical line type more visually prominent than the first graphical line type.

43. The medical system of claim 1, wherein the data processing device system is configured by the program at least to cause, via the input-output device system, a visual display of a third portion of the electrogram set just prior to the increasing of the visual prominence of the second portion of the electrogram set, wherein:
no electrogram in the third portion of the electrogram set is provided by any electrogram in the second portion of the electrogram set,
each electrogram of the third portion of the electrogram set is displayed according to a first visual characteristic set just prior to the increasing of the visual prominence of the second portion of the electrogram set, and
each electrogram of the third portion of the electrogram set is displayed according to a second visual characteristic set upon the increasing of the visual prominence of the second portion of the electrogram set, the second visual characteristic set less visually prominent than the first visual characteristic set.

44. The medical system of claim 43, wherein each electrogram of the third portion of the electrogram set is displayed with (i) a first degree of opacity according to the first visual characteristic set, and (ii) a second degree of opacity according to the second visual characteristic set, the second degree of opacity being less that the first degree of opacity.

45. The medical system of claim 43, wherein at least one transducer in the first transducer set is configured to record an electrogram, and the third portion of the electrogram set includes an electrogram recorded by the at least one transducer in the first transducer set.

46. The medical system of claim 1, wherein the data processing device system is configured by the program at least to cause, via the input-output device system, (a), (b), or (a) and (b) at least in response to the determination of the second transducer set.

47. A method executed by a data processing device system according to a program stored by a communicatively connected memory device system, the data processing device system also communicatively connected to an input-output device system, and the method comprising:
receiving input via the input-output device system indicating a selection of a first transducer set of a plurality of transducers of at least a portion of a transducer-based device, the at least the portion of the transducer-based device positionable within a bodily cavity, each transducer in the first transducer set configured to transmit tissue ablative energy;
determining, at least in response to the selection of the first transducer set, a second transducer set of the plurality of transducers satisfying a determined positional relationship with respect to the first transducer set, each transducer in the second transducer set configured to record a respective electrogram, and the second transducer set mutually exclusive with the first transducer set;
causing, via the input-output device system, display of at least part of an electrogram set; and
causing, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).

48. One or more non-transitory computer-readable storage mediums storing a program executable by a data processing device system communicatively connected to an input-output device system, the program comprising:
reception instructions configured to cause reception of input via the input-output device system indicating a selection of a first transducer set of a plurality of transducers of at least a portion of a transducer-based device, the at least the portion of the transducer-based device positionable within a bodily cavity, each transducer in the first transducer set configured to transmit tissue ablative energy;
determination instructions configured to cause determination, at least in response to the selection of the first transducer set, of a second transducer set of the plurality of transducers satisfying a determined positional relationship with respect to the first transducer set, each transducer in the second transducer set configured to record a respective electrogram, and the second transducer set mutually exclusive with the first transducer set;
first display instructions configured to cause, via the input-output device system, display of at least part of an electrogram set; and
second display instructions configured to cause, via the input-output device system, (a) a visual display of a first portion of the electrogram set in a state in which the first portion of the electrogram set was not visually displayed just prior to the visual display of the first portion of the electrogram set, each electrogram in the first portion of the electrogram set derived from or corresponding to at least one electrogram recorded by at least one respective transducer in the second transducer set, (b) an increasing of a visual prominence of a second portion of the electrogram set in a state in which the second portion of the electrogram set was visually displayed just prior to the increasing of the visual prominence of the second portion of the electrogram set, each electrogram in the second portion of the electrogram set derived from or corresponding to at least one electrogram recorded by at least one respective transducer in the second transducer set, or (a) and (b).
